(19) United States
(12) Patent Application Publication
(10) Pub. No.: US 2022/0367017 A1
(43) Pub. Date: Nov. 17, 2022

(54) HEALTH DATA MANAGEMENT METHODS, HEALTH DATA MANAGEMENT DEVICE AND BACKGROUND DEVICE

(71) Applicant: BOE TECHNOLOGY GROUP CO., LTD., Beijing (CN)

(72) Inventors: Chaozheng LIU, Beijing (CN); Guangyu SHAO, Beijing (CN); Xuewei YANG, Beijing (CN); Jiaao LI, Beijing (CN); Yangyi XU, Beijing (CN); Fuying DAI, Beijing (CN)

(73) Assignee: BOE TECHNOLOGY GROUP CO., LTD., Beijing (CN)

(21) Appl. No.: 17/765,759

(22) PCT Filed: Apr. 8, 2021
(86) PCT No.: PCT/CN2021/086028

§ 371 Date: Mar. 31, 2022

(30) Foreign Application Priority Data
May 29, 2020 (CN) 202010478397.0

(51) Int. Cl. G16H 10/60

(52) U.S. Cl. CPC G16H 10/60

(57) ABSTRACT

Health data management methods, a health data management device and a background device are provided. A health data management device can perform unified storage and unified processing for health data from different background devices by using the same data format, and health data of the same health type may have the same health index identifier. Therefore, the health data in different background devices may be interconnected, thereby improving flexibility and efficiency of use of the health data, reducing development costs, and improving development efficiency.

### CROSS-REFERENCE TO RELATED APPLICATIONS

This application is a national phase entry under 35 USC 371 of International Patent Application No. PCT/CN2021/086028 filed on Apr. 8, 2021, which claims priority to Chinese Patent Application No. 202010478397.0, filed on May 29, 2020, which are incorporated herein by reference in their entirety.

### TECHNICAL FIELD

The present disclosure relates to the field of electronic technologies, and in particular, to health data management methods, a health data management device and a background device.

### BACKGROUND

People's health awareness has improved significantly with the development of society. At present, there are various health data collection devices on the market, such as wristbands, sleep instruments, sports watches. Generally, the health data collected by a health data collection device is stored in a background device that provides service therefor.

Different health data collection devices may correspond to different background devices. Storage rules, query rules and processing rules of different background devices may be different.

### SUMMARY

In a first aspect, a health data management method is provided. The method includes: receiving and storing first health data sent by a first background device, the first health data including a first health index identifier and a first data content corresponding to the first health index identifier; and receiving and storing second health data sent by a second background device, the second health data including a second health index identifier and a second data content corresponding to the second health index identifier. Data formats of the first health data and the second health data are same. In a case where health types of the first data content and the second data content are same, the first health index identifier and the second health index identifier are same. In a case where the health types of the first data content and the second data content are different, the first health index identifier and the second health index identifier are different.

In some embodiments, before receiving and storing the first health data sent by the first background device, the method further includes: registering the first background device according to a received first registration request, and returning first information to the first background device. The first information includes an identifier of the first background device and a key of the first background device. And/or, before receiving and storing the second health data sent by the second background device, the method further includes: registering the second background device according to a received second registration request, and returning second information to the second background device. The second information includes an identifier of the second background device and a key of the second background device.

In some other embodiments, registering the first background device according to the received first registration request includes: displaying a first interface, the first interface having a first control thereon; determining a first preset operation for the first control on the first interface; displaying a second interface in response to the first preset operation; and receiving information of the first background device input to the second interface, so as to generate the identifier of the first background device and the key of the first background device. And/or, registering the second background device according to the received second registration request includes: displaying a first interface, the first interface having a first control thereon; determining a first preset operation for the first control on the first interface; displaying a second interface in response to the first preset operation; and receiving information of the second background device input to the second interface, so as to generate the identifier of the second background device and the key of the second background device.

In some other embodiments, if a health index identifier corresponding to a health type of first health information is not provided, registering the first background device according to the received first registration request further includes: generating the first health index identifier corresponding to the health type of the first health information according to the received first registration request, the health type of the first health information being a health type of health information that is able to be collected by a health data collection device for which the first background device provides service. And/or, if a health index identifier corresponding to a health type of second health information is not provided, registering the second background device according to the received second registration request further includes: generating the second health index identifier corresponding to the health type of the second health information according to the received second registration request, the health type of the second health information being a health type of health information that is able to be collected by a health data collection device for which the second background device provides service.

In some other embodiments, generating the first health index identifier corresponding to the health type of the first health information according to the received first registration request includes: displaying a third interface, the third interface having a second control thereon; determining a second preset operation for the second control on the third interface; displaying a fourth interface in response to the second preset operation; and receiving third information related to the health type of first health information input to the fourth interface, so as to generate the first health index identifier corresponding to the health type of the first health information.

In some other embodiments, generating the second health index identifier corresponding to the health type of the second health information according to the received second registration request includes: displaying the third interface, the third interface having the second control thereon; determining the second preset operation for the second control on the third interface; displaying the fourth interface in response to the second preset operation; and receiving fourth information related to the health type of second health information input to the fourth interface, so as to generate the second health index identifier corresponding to the health type of the second health information.

In some other embodiments, after receiving and storing the second health data sent by the second background device, the method further includes: in a case where a first user identifier and a second user identifier are same, performing data processing in combination with the first data content and the second data content to obtain a data processing result.

In some other embodiments, performing data processing in combination with the first data content and the second data content to obtain the data processing result includes: in a case where data types of the first data content and the second data content are both numbers, performing a weighted average processing on the first data content and the second data content to obtain the data processing result; or, in a case where the data types of the first data content and the second data content are both texts, deleting a part, having the same semantics in the first data content and the second data content, of the first data content or the second data content.

In some other embodiments, after performing data processing in combination with the first data content and the second data content to obtain the data processing result, the method further includes: transmitting the data processing result to the first background device; and/or, transmitting the data processing result to the second background device; and/or, transmitting the data processing result to a presentation device.

In some other embodiments, after receiving and storing the second health data sent by the second background device, the method further includes: receiving a first data request from the first background device, the first data request being configured to request health data of a first user, and the first data request including an identifier of the first background device and a key of the first background device; authenticating the first background device in response to the first data request; and transmitting the first health data to the first background device if authentication of the first background device passes.

In some other embodiments, the method further includes: in a case where a first user identifier and a second user identifier are same, transmitting the second health data to the first background device if the authentication of the first background device passes.

In a second aspect, a health data management method is provided. The method includes: receiving first health information collected by a first health data collection device and sent by a first terminal, the first health information including first user information and a first data content; generating first health data based on the first health information, a configured first health index identifier corresponding to a health type of the first data content, and fourth information, the fourth information being configured to indicate a data format of health data, the first health data including a first user identifier, a first data content and a first health index identifier, and the first user information and the first user identifier being used to represent a same user; and sending the first health data to a health data management device.

In some embodiments, after sending the first health data to the health data management device, the method further includes: receiving a first data request from the first terminal, the first data request being configured to request health data of a first user, and the first data request including an identifier of the first background device and a key of the first background device; sending the first data request to the health data management device; and receiving the first health data from the health data management device.

In some other embodiments, in a case where the first user identifier is same as a second user identifier of second health data, after sending the first data request to the health data management device, the method further includes: receiving the second health data from the health data management device. The second health data is health data from a second background device and stored in the health data management device, and the second health data includes the second user identifier, a second health index identifier and a second data content corresponding to the second health index identifier.

In some other embodiments, after receiving the first health data from the health data management device, the method further includes: caching the first health data.

In a third aspect, a health data management method is provided. The method includes: receiving, by a health data management device, a first registration request, the first registration request being configured to instruct to register a first background device; generating, by the health data management device, a first health index identifier according to the received first registration request if a health index identifier corresponding to a health type of first health information is not provided, wherein the first health index identifier is configured to identify the health type of the first health information, and the health type of the first health information is a health type of health information that is able to be collected by a health data collection device for which the first background device provides service; receiving, by the first background device, the first health index identifier and fourth information, the fourth information being configured to indicate a data format of health data; generating, by the first background device, first health data based on the first health information, the first health index identifier and the fourth information, the first health data including the first health index identifier; and sending, by the first background device, the first health data to the health data management device.

In some embodiments, the method further includes: receiving, by the health data management device, a second registration request, the second registration request being configured to instruct to register a second background device; generating, by the health data management device, a second health index identifier according to the received second registration request if a health index identifier corresponding to a health type of second health information is not provided, wherein the second health index identifier is configured to identify the health type of the second health information, and the health type of the second health information is a health type of health information that is able to be collected by a health data collection device for which the second background device provides service; receiving, by the second background device, the second health index identifier and the fourth information, the fourth information being configured to indicate the data format of the health data; generating, by the second background device, second health data based on the second health information, the second health index identifier and the fourth information, the second health data including the second health index identifier; and sending, by the second background device, the second health data to the health data management device.

In some other embodiments, the method further includes: sending, by the first background device, a first data request to the health data management device, the first data request being configured to request health data of a first user, and the first data request including an identifier of the first background device and a key of the first background device; authenticating, by the health data management device, the first background device in response to the first data request; transmitting, by the health data management device, the first health data to the first background device if authentication of the first background device passes; and in a case where the first health index identifier and the second health index identifier are same, further transmitting, by the health data management device, the second health data to the first background device if the authentication of the first background device passes.

In a fourth aspect, a health data management device is provided. The health data management device includes a transceiver unit and a storage unit. The transceiver unit is used to receive first health data sent by a first background device, wherein the first health data includes a first user identifier, a first health index identifier, and a first data content corresponding to the first health index identifier. The transceiver unit is further used to receive second health data sent by a second background device, wherein the second health data includes a second user identifier, a second health index identifier, and a second data content corresponding to the second health index identifier. Data formats of the first health data and the second health data are same. In a case where health types of the first data content and the second data content are same, the first health index identifier and the second health index identifier are same. In a case where the health types of the first data content and the second data content are different, the first health index identifier and the second health index identifier are different. The storage unit is used to store the first health data and the second health data.

In some embodiments, the health data management device further includes a processing unit, and the processing unit includes an external system management module. The external system management module is used to: display a first interface, the first interface having a first control thereon; determine a first preset operation for the first control on the first interface; display a second interface in response to the first preset operation; and receive information of the first background device input to the second interface, so as to generate an identifier of the first background device and a key of the first background device; and/or, display a first interface, the first interface having a first control thereon; determine a first preset operation for the first control on the first interface; display a second interface in response to the first preset operation; and receive information of the second background device input to the second interface, so as to generate an identifier of the second background device and a key of the second background device.

In some other embodiments, the processing unit further includes an index management module. The index management module is used to: display a third interface, the third interface having a second control thereon; determine a second preset operation for the second control on the third interface; display a fourth interface in response to the second preset operation; and receive third information related to a health type of first health information input to the fourth interface, so as to generate the first health index identifier corresponding to the health type of the first health information; and/or, display a third interface, the third interface having a second control thereon; determine a second preset operation for the second control on the third interface; display a fourth interface in response to the second preset operation; and receive fourth information related to a health type of the second health information input to the fourth interface, so as to generate the second health index identifier corresponding to the health type of the second health information.

In some other embodiments, the processing unit is used to, in a case where the first health index identifier and the second health index identifier are the same, and the first user identifier and the second user identifier are same, perform data processing in combination with the first data content and the second data content to obtain a data processing result.

In some other embodiments, the transceiver unit further includes a unified data transmission interface and an external interface. The unified data transmission interface is used to: transmit the data processing result to the first background device; and/or, transmit the data processing result to the second background device. The external interface is used to transmit the data processing result to a presentation device.

In some other embodiments, the transceiver unit is further used to: receive a first data request from the first background device, wherein the first data request is configured to request health data of a first user, and the first data request includes the identifier of the first background device and the key of the first background device. The processing unit is further used to: authenticate the first background device in response to the first data request; and transmit the first health data to the first background device if authentication of the first background device passes.

In some other embodiments, the unified data transmission interface is further used to, in the case where the first user identifier and the second user identifier are the same, transmit the second health data to the first background device if the authentication of the first background device passes.

In a fifth aspect, a background device is provided. The background device includes a transceiver unit and a processing unit. The transceiver unit is used to receive first health information collected by a first health data collection device and sent by a first terminal, the first health information includes first user information and a first data content. The processing unit is used to generate first health data based on the first health information, a configured first health index identifier corresponding to a health type of the first data content, and fourth information, wherein the fourth information is used to indicate a data format of health data, the first health data includes a first user identifier, the first data content and the first health index identifier, the first user information and the first user identifier are used to represent a same user. The transceiver unit is further used to send the first health data to a health data management device.

In some embodiments, the transceiver unit is further used to: receive a first data request from the first terminal, the first data request being configured to request health data of a first user, and the first data request including an identifier of a background device and a key of a background device; send the first data request to the health data management device; and receive the first health data from the health data management device.

In some other embodiments, in a case where the first user identifier is the same as a second user identifier of second health data, the transceiver unit is further used to: receive the second health data from the health data management device, wherein the second health data is health data from another background device other than the background device and stored in the health data management device, and the second health data includes the second user identifier, a second health index identifier, and a second data content corresponding to the second health index identifier.

In a sixth aspect, a health data management system is provided. The health data management system includes a health data management device and at least two background devices. The health data management device is the health data management device as described in any of the above contents in the fourth aspect, and a background device is the background device as described in any of the above contents in the fifth aspect.

In a seventh aspect, a health data management device is provided. The health data management device includes a processor and a memory. The memory is used to store computer instructions that, when executed by the processor, cause the health data management device to perform the health data management method as described in any of the above contents in the first aspect.

In an eighth aspect, a background device is provided. The background device includes a processor and a memory. The memory is used to store computer instructions that, when executed by the processor, cause the background device to perform the health data management method as described in any of the above contents in the second aspect.

In a ninth aspect, a computer-readable storage medium is provided. The computer-readable storage medium has stored thereon computer program instructions that, when run on a computer (e.g., a health data management device), cause the computer to perform one or more steps of the health data management method as described in any of the above contents in the first aspect.

In a tenth aspect, a computer-readable storage medium is provided. The computer-readable storage medium has stored thereon computer program instructions that, when run on a computer (e.g., a background device), cause the computer to perform one or more steps of the health data management method as described in any of the above contents in the second aspect.

In an eleventh aspect, a computer program product is provided. The computer program product includes computer program instructions that, when executed by a computer (e.g., a health data management device), cause the computer to perform one or more steps of the health data management method as described in any of the above contents in the first aspect.

In a twelfth aspect, a computer program product is provided. The computer program product includes computer program instructions that, when executed by a computer (e.g., a background device), cause the computer to perform one or more steps of the health data management method as described in any of the above contents in the second aspect.

In a thirteenth aspect, a computer program product is provided. The computer program product includes computer program instructions that, when executed by a computer, cause the computer to perform one or more steps of the health data management method as described in any of the above contents in the third aspect.

In a fourteenth aspect, a computer program is provided. When executed by a computer (e.g., a health data management device), the computer program causes the computer to perform one or more steps of the health data management method as described in any of the above contents in the first aspect.

In a fifteenth aspect, a computer program is provided. When executed by a computer (e.g., a background device), the computer program causes the computer to perform one or more steps of the health data management method as described in any of the above contents in the second aspect.

In a sixteenth aspect, a computer program is provided. When executed by a computer, the computer program causes the computer to perform one or more steps of the health data management method as described in any of the above contents in the third aspect.

### BRIEF DESCRIPTION OF THE DRAWINGS

In order to describe technical solutions in the present disclosure more clearly, accompanying drawings to be used in some embodiments of the present disclosure will be introduced briefly below. Obviously, the accompanying drawings to be described below are merely accompanying drawings of some embodiments of the present disclosure, and a person of ordinary skill in the art can obtain other drawings according to these drawings. In addition, the accompanying drawings in the following description may be regarded as schematic diagrams, and are not limitations on actual sizes of products, actual processes of methods and actual timings of signals involved in the embodiments of the present disclosure.

FIG. 1A is a structural diagram of a health data management system, in accordance with some embodiments of the present disclosure;

FIG. 1B is a structural diagram of a health data management device, in accordance with some embodiments of the present disclosure;

FIG. 2A is a schematic diagram of an interface of a health data management device, in accordance with some embodiments of the present disclosure;

FIG. 2B is a schematic diagram of another interface of a health data management device, in accordance with some embodiments of the present disclosure;

FIG. 2C is a schematic diagram of yet another interface of a health data management device, in accordance with some embodiments of the present disclosure;

FIG. 2D is a schematic diagram of yet another interface of a health data management device, in accordance with some embodiments of the present disclosure;

FIG. 2E is a schematic diagram of yet another interface of a health data management device, in accordance with some embodiments of the present disclosure;

FIG. 2F is a schematic diagram of yet another interface of a health data management device, in accordance with some embodiments of the present disclosure;

FIG. 2G is a schematic diagram of yet another interface of a health data management device, in accordance with some embodiments of the present disclosure;

FIG. 2H is a schematic diagram of yet another interface of a health data management device, in accordance with some embodiments of the present disclosure;

FIG. 2I is a schematic diagram of an index of health data, in accordance with some embodiments of the present disclosure;

FIG. 3A is a structural diagram of a computer system, in accordance with some embodiments of the present disclosure;

FIG. 3B is a flow diagram of a health data management method, in accordance with some embodiments of the present disclosure;

FIG. 4 is a flow diagram of another health data management method, in accordance with some embodiments of the present disclosure;

FIG. 5A is a flow diagram of a method for registering a first background device, in accordance with some embodiments of the present disclosure;

FIG. 5B is a flow diagram of a method for registering a second background device, in accordance with some embodiments of the present disclosure;

FIG. 5C is a flow diagram of another method for registering a first background device, in accordance with some embodiments of the present disclosure;

FIG. 5D is a flow diagram of another method for registering a second background device, in accordance with some embodiments of the present disclosure;

FIG. 6 is a schematic diagram of a displayed data processing result, in accordance with some embodiments of the present disclosure;

FIG. 7 is a flow diagram of yet another health data management method, in accordance with some embodiments of the present disclosure;

FIG. 8 is a flow diagram of yet another health data management method, in accordance with some embodiments of the present disclosure;

FIG. 9 is a flow diagram of yet another health data management method, in accordance with some embodiments of the present disclosure;

FIG. 10 is a flow diagram of yet another health data management method, in accordance with some embodiments of the present disclosure;

FIG. 11 is a flow diagram of yet another health data management method, in accordance with some embodiments of the present disclosure;

FIG. 12 is a flow diagram of yet another health data management method, in accordance with some embodiments of the present disclosure;

FIG. 13 is a structural diagram of a background device, in accordance with some embodiments of the present disclosure; and

FIG. 14 is a connection diagram of a health data management device and a presentation device, in accordance with some embodiments of the present disclosure.

### DETAILED DESCRIPTION

Technical solutions in some embodiments of the present disclosure will be described clearly and completely below with reference to the accompanying drawings. Obviously, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as open and inclusive, i.e., “including, but not limited to”. In the description of the specification, the terms such as “one embodiment”, “some embodiments”, “exemplary embodiments”, “example”, “specific example” and “some examples” are intended to indicate that specific features, structures, materials or characteristics related to the embodiment(s) or example(s) are included in at least one embodiment or example of the present disclosure. Schematic representations of the above terms do not necessarily refer to the same embodiment(s) or example(s). In addition, the specific features, structures, materials or characteristics may be included in any one or more embodiments or examples in any suitable manner.

Hereinafter, terms such as “first” and “second” are only used for descriptive purposes, and are not to be construed as indicating or implying relative importance or implicitly indicating the number of indicated technical features. Thus, features defined by “first” or “second” may explicitly or implicitly include one or more of the features. In the description of the embodiments of the present disclosure, the term “a plurality of” or “the plurality of” means two or more unless otherwise specified.

The phrase “at least one of A, B and C” has the same meaning as the phrase “at least one of A, B or C”, and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

As used herein, the term “if” is optionally construed as “when” or “in a case where” or “in response to determining that” or “in response to detecting”, depending on the context. Similarly, the phrase “if it is determined” or “if [a stated condition or event] is detected” is optionally construed as “in a case where it is determined”, “in response to determining”, “in a case where [the stated condition or event] is detected” or “in response to detecting [the stated condition or event]”, depending on the context.

The use of the phase “applicable to” or “configured to” herein means an open and inclusive language, which does not exclude devices that are applicable to or configured to perform additional tasks or steps.

At present, health data obtained by a health data collection device on the market are usually stored in a corresponding background device. Different health data collection devices may correspond to different background devices, and storage rules, query rules and processing rules of these background devices may be different. As a result, health data stored in different background devices are unable to interconnect, thus it is difficult to perform unified processing, thereby reducing flexibility and efficiency of use of these data, increasing developing costs and lowering developing efficiency. In addition, since different background devices may have different processing rules for health data, the result obtained by a user through different background devices may also be different, which reduces the user's use experience.

The embodiments of the present disclosure provide a health data management system, in which a health data management device can perform unified storage and unified processing for the health data from different background devices by using a same data format, and health data of the same health type may have the same health index identifier. Each background device may provide service for at least one health data collection device. For example, each background device may receive health data from corresponding health data collection device(s), and analyze and process the health data.

Based on the health data management system provided by the embodiments of the present disclosure, it is possible to perform the unified storage and the unified processing for the health data, the health data in different background devices are able to interconnect, thereby improving the flexibility and the efficiency of the use of the health data. Compared with the technical solution, in the related art, that various background devices each manage a respective health data, the developing costs may be reduced, the developing efficiency may be improved, and the labor costs may be reduced.

In addition, the unified processing is based on the health data from different background devices rather than the health data from a single background device (i.e., the unified processing is based on whole data instead of single data), and the unified processing result can be transmitted to the background devices for result display. As a result, accuracy and reliability of the processed result may increase, and the user's user experience may also be improved.

Moreover, since health data from different background devices are stored in a same data format, each background device does not need to store a large amount of health data separately, which may reduce requirements for a memory of the background device and help improve the efficiency of the background device.

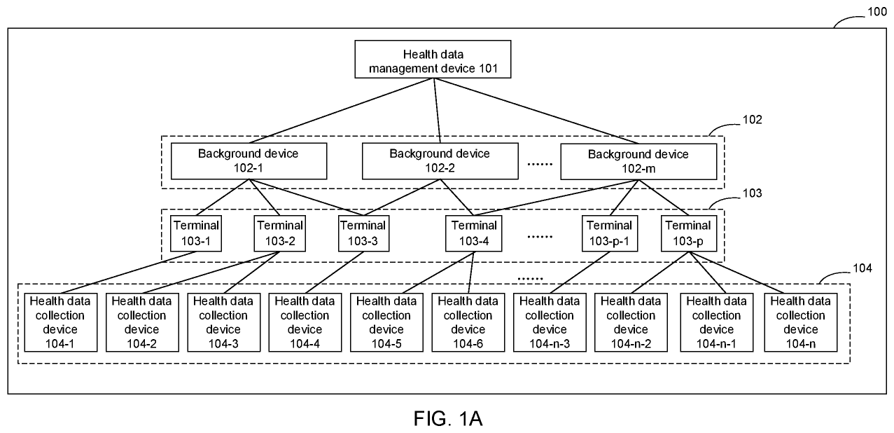

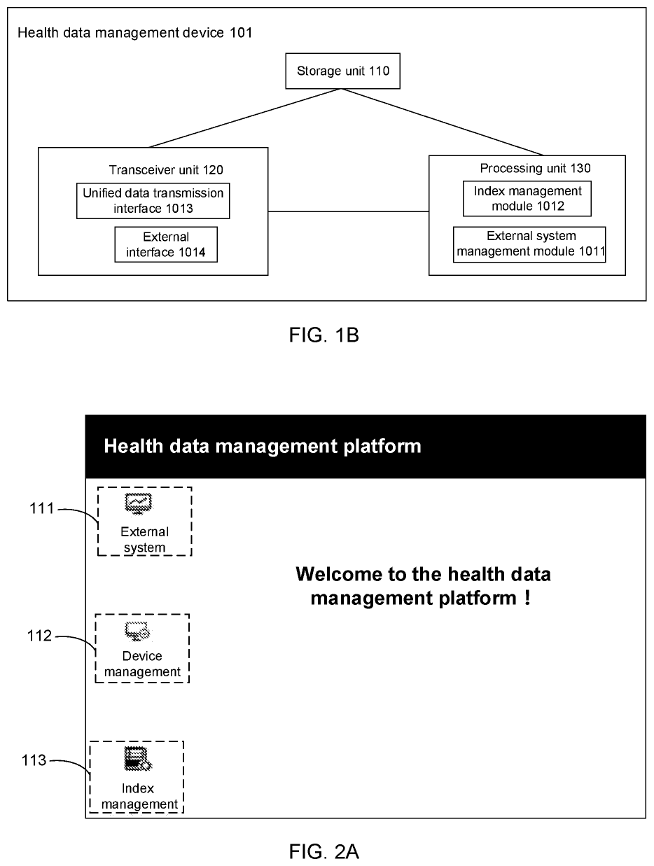

For example, FIG. 1A is a structural diagram of a health data management system, in accordance with some embodiments of the present disclosure. Referring to FIG. 1A, the health data management system 100 may include a health data management device 101, background devices 102, terminals 103 and health data collection devices 104.

The health data management device 101 may include at least one health data management server. In a case where the health data management device 101 includes a plurality of (at least two) health data management servers, the health data management device 101 may be called as a server cluster. The health data management device 101 may also be a health data management platform (i.e., a software system). A background device 102 may be a background server or an external system (a hardware system or a software system). A health data collection device 104 may be referred to simply as a “collection device” or a “device”.

In addition, there may be one or more background devices 102, terminals 103 and health data collection devices 104. For example, the background devices may include a background device 102-1, a background device 102-2 and a background device 102-3 that are shown in FIG. 1A. The terminals may include a terminal 103-1, a terminal 103-2, a terminal 103-3, a terminal 103-4, a terminal 103-(p-1) and a terminal 103-p that are shown in FIG. 1A. The health data collection devices may include a health data collection device 104-1, a health data collection device 104-2, a health data collection device 104-3, a health data collection device 104-4, . . . , a health data collection device 104-(n-1), a health data collection device 104-n that are shown in FIG. 1A.

Each background device 102 may provide service for at least one health data collection device 104. For example, the health data collected by the health data collection device 104 may be transmitted to a corresponding background device 102 through a terminal 103. Each background device may correspond to at least one terminal. For example, referring to FIG. 1A, the background device 102-1 corresponds to three terminals, i.e., the terminal 103-1, the terminal 103-2 and the terminal 103-3. Each terminal can be connected to at least one background device for information exchange related to health data. For example, referring to FIG. 1A, the terminal 103-4 may be connected to the background device 102-2 and the background device 102-m. Each terminal may be connected to at least one health data collection device, so as to transmit the health information collected by the at least one health data collection device to the terminal, and then transmit the health information to corresponding background device(s) through the terminal. For example, referring to FIG. 1A, the terminal 103-4 may be connected to the health data collection device 104-5 and the health data collection device 104-6, so as to transmit the health information collected by the health data collection device 104-5 to the background device 102-2, and transmit the health information collected by the health data collection device 104-6 to the background device 102-m.

For example, the health data collected by the health data collection device 104 is not limited to being transmitted to the corresponding background device 102 through the terminal 103 as shown in FIG. 1, and the health data collected by the health data collection device 104 may also be directly transmitted to the corresponding background device 102.

Different background devices can transmit health data from different health data collection devices to the health data management device for storage by using a unified data format. For example, the unified data format may be a json format. For another example, the unified data format may be an XML format. For yet another example, the unified data format may be a CSV format. Of course, the unified data format may also be other types of data formats, and a type of the unified data format is not limited in the embodiments of the present disclosure.

In addition, the health data of the same health type (e.g., heartbeat and pulse) may have the same health index identifier. That is, the same health index identifier is set for the health data, of the same health type, detected by different health data collection devices, and the health index identifier may also be referred to as a health index code. For example, the health index identifier of diastolic pressure may be DE04.10.176.00, the health index identifier of systolic pressure may be DE04.10.174.00, the health index identifier of glycated hemoglobin may be DE04.50.083.00, and the health index code of sleep may be DE04.60.063.00. If both a sleep instrument and a sports watch have sleep data, the health index identifiers of the sleep data thereof are the same, so that all sleep data, from the sleep instrument and the sports watch, stored in the health data management device 101 have the same health index identifier, which is conducive to performing the unified management of sleep data for the health data management device 101.

Since the health data management device 101 stores health data by using a unified data format, and the health data of the same health type may have the same health index identifier, the health data management device 101 may aggregate health data from different background devices for analysis and processing, so as to improve the accuracy and the reliability of processing the health data, and improve the user's use experience. In addition, the unified management of data, compared with the technical solution, in the related art, that health data are managed separately by various background devices, may reduce the developing workload significantly, improve the developing efficiency, and reduce the labor costs.

In addition, referring to FIG. 1B, the health data management device 101 may include a storage unit 110, a transceiver unit 120 and a processing unit 130.

The storage unit 110 may be used to help the health data management device 101 store program codes, data, and the like. For example, the storage unit 110 may store the health data provided by the embodiments of the present disclosure. The transceiver unit 120 may be used to receive data (e.g., health data and processing result of health data) from other devices (e.g., background devices) and send data to other devices. The processing unit 130 may be used to control and manage the actions of the health data management device 101. For example, the processing unit 130 may be used to help the health data management device 101 perform the steps performed by the storage unit 110 and the transceiver unit 120.

The processing unit 130 includes an external system management module 1011 and an index management module 1012. The health data management device 101 can register (also referred to as “create”) the background devices through the external system management module 1011, and the registration information may include a code of the background device, a name of the background device, an identifier of the background device, a key of the background device, an introduction of the background device, etc. After the registration successes, the external system management module 1011 can return the identifier of the background device and the key of the background device that are corresponding to the background device to the background device 102.

The health index identifiers of various types of health data transmitted by the background devices to the health data management device 101 need to be entered into the index management module 1012 first, so that the health data of the same health type from different background devices have the same health index identifier, thereby facilitating the unified management of the health data.

The transceiver unit 120 includes a unified data transmission interface 1013 and an external interface 1014.

The unified data transmission interface 1013 shown in FIG. 1B is an interface for data transmission between the background device and the health data management device 101. Through the unified data transmission interface 1013, the health data management device 101 can receive health data with a unified data format from different background devices 102, and can also send corresponding health data with the unified data format to a background device 102 in response to the request of the background device 102.

The external interface 1014 is an interface for data transmission between the health data management device 101 and another device other than the background device.

In some implementations, the health data management device 101 may transmit the processing result to the background device 102 through the unified data transmission interface 1013, and then the background device 102 transmits the processing result to the corresponding terminals 103 for display. That is, as front ends of the background device 102, the terminals 103 may be used to receive and display the processing result from the background device 102. One background device 102 may correspond to one or more terminals 103. In order to simplify the description, one background device 102 corresponds to one terminal 103.

In some other implementations, as shown in FIG. 14, the health data management device 101 can also transmit the processing result to a presentation device 105 for display through the external interface 1014. The presentation device may be a device other than the terminal 103 shown in FIG. 1A. For example, the presentation device may be a mobile phone, a tablet computer or a desktop computer, and a type of the presentation device is not limited in the embodiments of the present disclosure.

In some other implementations, the health data management device 101 may also directly provide a display page to display the processing result.

It will be understood that, the method in which the health data management device 101 displays the processing result of the health data is not limited to the above examples, and there may also be other display methods. The method in which the health data management device 101 displays the processing result of the health data is not limited in the embodiments of the present disclosure.

In addition, the health data management device 101 can send corresponding health data to the background device 102 in response to the request of the background device 102, and the background device 102 can process the health data and display the processing result through corresponding terminal(s) 103.

The health data management device 101 stores the health data by using a unified data format, and the health data of the same health type (e.g., heartbeat and pulse) have the same health index identifier. Therefore, when a certain background device 102 requests the health data of a type b of a user a from the health data management device 101, in addition to sending the health data of the type b of the user a received from the background device 102 itself to the background device 102, the health data management device 101 may also send the health data of the type b of the user a from other background devices 102 to the background device 102. Thus, in addition to the health data of the type b of the user a from the background device 102 to the health data management device 101, the background device 102 can also receive the health data of type b of the user a from other background devices 102, so that the background device 102 may aggregate the health data of the type b of the user a from different background devices 102 for analysis and processing, which may help improve the accuracy and the reliability of the processing result.

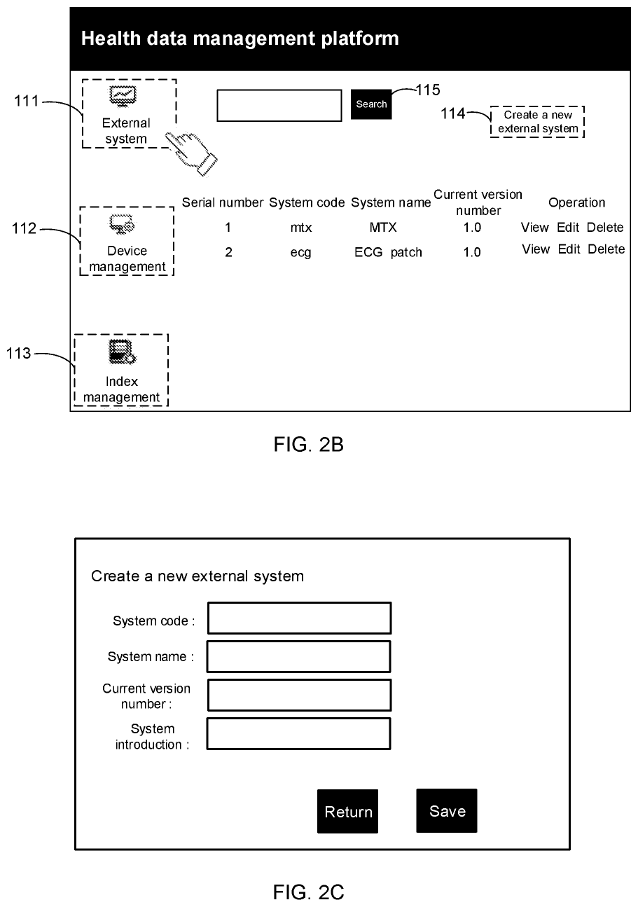

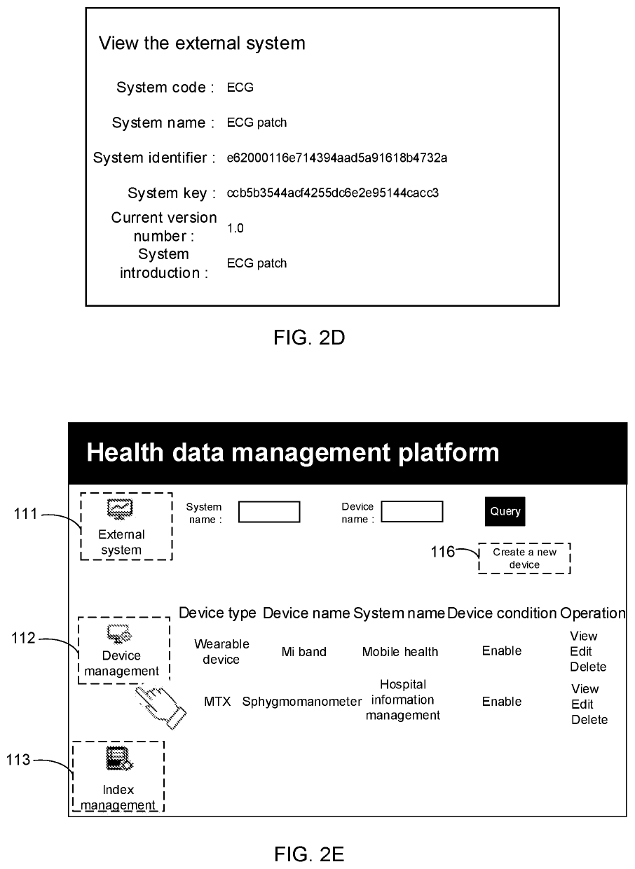

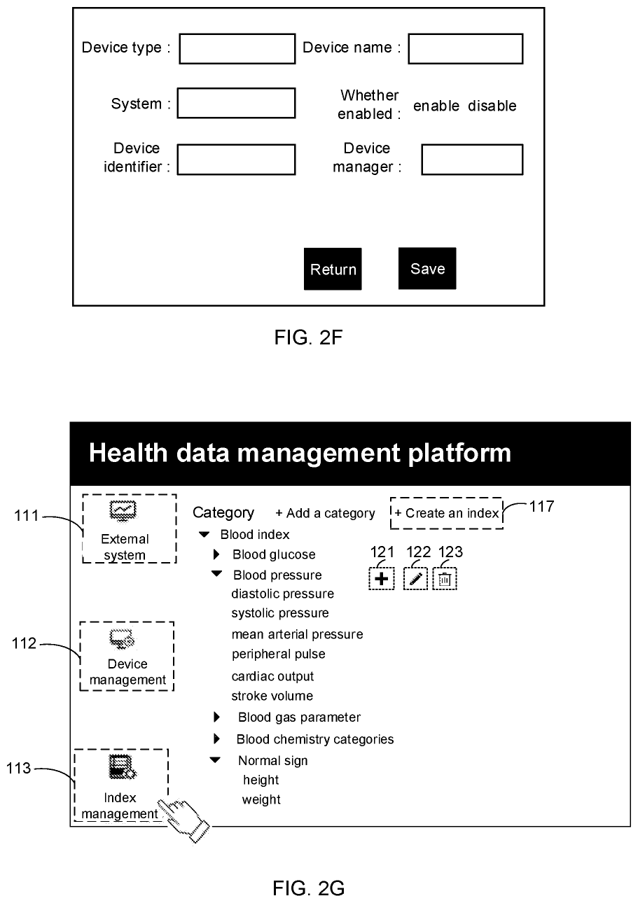

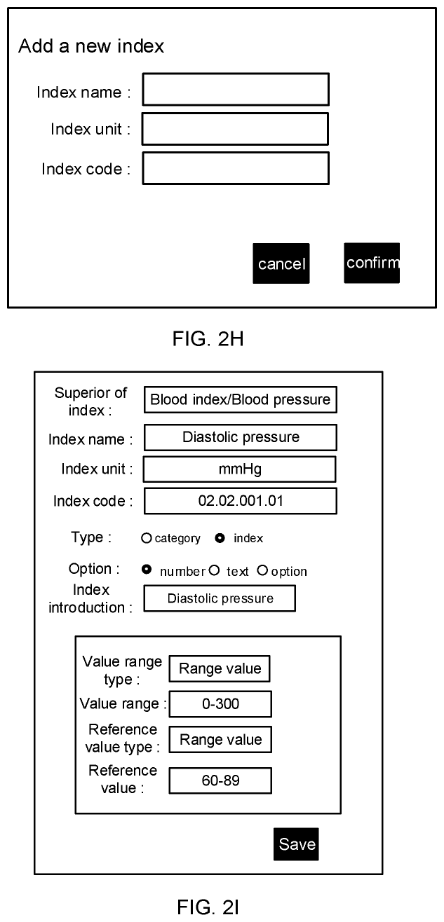

For example, the health data management device may have an interactive interface shown in FIG. 2A. The interactive interface may also be referred to as a main interface. As shown in FIG. 2A, the interface may include a control of “External system” 111, a control of “Device management” 112 and a control of “Index management” 113.

As mentioned above, each background device can provide service for at least one health data collection device, a plurality of health data collection devices can collect health data of various health types, and the health data of the same health type may have the same health index identifier. Thus, when creating a background device, in addition to creating the background device itself, it may also need to create health data collection device(s) for which the background device provides service, and health indexes of the health data corresponding to the health data collection device(s). For example, a background device, a health data collection device for which the background device provides service, and a health index of the health data corresponding to the health data collection device may be created through the control of the “External system” 111, the control of the “Device management” 112 and the control of the “Index management” 113 that are shown in FIG. 2A, respectively.

The process of creating the background device will be described below. For example, an administrator of the health data management device may click the control of the “External system” 111 shown in FIG. 2A to enter an “External system” interface shown in FIG. 2B. Then, the administrator clicks a control of “Create a new external system” 114 in the “External system” interface to enter a “Create a new external system” interface shown in FIG. 2C, and input specific information of a system code, a system name, a current version number and a system introduction in the “Create a new external system” interface and click “Save”, thereby creating a new external system. For example, the specific information of the system code, the system name, the current version number and the system introduction may each be “ECG”, “Electrocardiogram (ECG) patch”, “1.0” and “ECG patch”.

In addition, the health data management device may automatically assign a system identifier and a system key to the created external system in response to the above operation of clicking “Save”.

Then, for example, the created external system may automatically be displayed on the “External system” interface. For another example, the created external system are not automatically displayed on the “External system” interface, and the created external system may be queried through a control of “Search” 115 shown in FIG. 2B. In addition, each of created external systems may be managed through controls of “View”, “Edit” and “Delete”. For example, a “View the external system” interface shown in FIG. 2D may be displayed by clicking a control of “View” corresponding to the external system whose system name is “ECG patch”. In the “View the external system” interface, in addition to the specific information of the system code, the system name, the current version number and the system introduction, the specific information of the system identifier and the system key automatically assigned to the created external system is also displayed.

The process of creating the health data collection device for which the background device provides service will be described below. For example, the administrator of the health data management device may click the control of the “Device management” 112 shown in FIG. 2A to enter a “Device management” interface shown in FIG. 2E. Then, the administrator may click a control of “Create a new device” 116 in the “Device management” interface to enter a “Create a new device” interface shown in FIG. 2F. A new device is created after inputting the specific information such as a device type, a device name, a device identifier in the “Create a new device” interface and clicking “Save”.

In addition, as shown in FIG. 2E, the created device may be displayed on the “Device management” interface, and the created device may be searched through a control of “Query”. In addition, each of created devices may be managed through controls of “View”, “Edit” and “Delete”.

The process of creating the health index of the health data corresponding to the health data collection device will be described below. First of all, it will be noted that, if the health data management server has created corresponding health indexes for the health types of all the health data corresponding to the health data collection devices for which the newly created background device provides service (i.e., the health types related to the health data have been created previously), there is no need to recreate them. If there are health type(s) that have not been created previously in the health types of the health data corresponding to the health data collection devices for which the newly created background device provides service, the health indexes of the health type(s) that have not been created previously are created.

For example, the administrator of the health data management device may click the control of the “Index management” 113 shown in FIG. 2A to enter an “Index management” interface shown in FIG. 2G. Then, the administrator may click a control of “Create an index” 117 in the “Index management” interface to enter a “Create an index” interface shown in FIG. 2H. A new index is created after inputting the specific information of an index name, an index unit, an index code in the “Create an index” interface and clicking “Yes”.

In addition, as shown in FIG. 2G, the created index may be displayed on the “Index management” interface, and the created index may be searched by the control of “Query”. In addition, each of created indexes may be managed through controls of “View”, “Edit” and “Delete”.

In addition, as shown in FIG. 2G, indexes may be classified into different directories and subdirectories according to categories. For example, the blood index directory includes subdirectories such as blood glucose, blood pressure, blood gas parameters and blood chemistry categories, and specific indexes in the blood pressure subdirectory includes diastolic pressure, systolic pressure, mean arterial pressure, peripheral pulse, etc. Through controls of “Add”, “Modify” and “Delete” corresponding to a directory (or a sub-directory), an index may be added into the directory (or the sub-directory), the directory (or the sub-directory) may be modified, and the directory (or the sub-directory) may be deleted. For example, for the blood pressure subdirectory shown in FIG. 2G, an index may be added into the blood pressure subdirectory, the blood pressure subdirectory may be modified, and the blood pressure subdirectory may be deleted through the control of “Add” 121, the control of “Modify” 122 and the control of “Delete” 123, respectively.

When viewing the created index, as an example, an index with its name of “Diastolic pressure” is show in FIG. 2I. Referring to FIG. 2I, the diastolic pressure is an index in the directory of “Blood index/Blood pressure”, thus a superior index of the index is “Blood index/Blood pressure”. The index name is diastolic pressure, and the index unit is mmHg (millimeter of mercury). The index code (i.e., the index identifier) is 02.02.001.01. A type is “index”, and an option for the index is “number”. Value range type and reference value type of the index are both “range value”, where the value range is in a range of 0 to 300, and the reference value is in a range of 60 to 89.

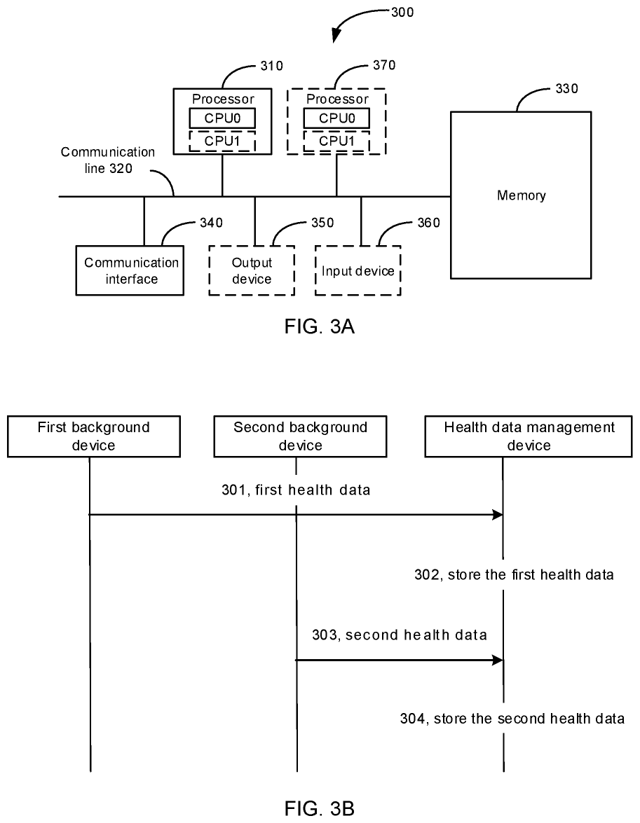

For example, both the health data management device 101 and the background device 102 in FIG. 1 may be implemented by a computer system 300 shown in FIG. 3A. The computer system 300 includes at least one processor 310, a communication line 320, a memory 330 and at least one communication interface 340.

The processor 310 may be a central processing unit (CPU), a microprocessor, an application specific integrated circuit (ASIC), or one or more integrated circuits used for controlling an implementation of the program of solutions in the embodiments of the present disclosure.

The communication line 320 may include a channel to communicate information between the components described above.

The communication interface 340 adopts devices such as a transceiver and is used to communicate with other devices or communication networks, such as Ethernet, radio access network (RAN), wireless local area networks (WLAN).

The memory 330 may be a read-only memory (ROM) or a static storage device of any other type that may store static information and instructions, a random access memory (RAM) or a dynamic storage device of any other type that may store information and instructions, or an electrically erasable programmable read-only memory (EEPROM), a compact disc read-only memory (CD-ROM) or any other compact disc storage or optical disc storage (including a compressed disc, a laser disc, an optical disc, a digital versatile disc or a Blu-ray disc), a magnetic disc storage medium or any other magnetic storage device, or any other medium that can be used to carry or store desired program codes in a form of instructions or data structures and that can be accessed by a computer, but it is not limited thereto. The memory may exist independently and is connected to the processor through a bus (i.e., the communication line 320). The memory may also be integrated with the processor(s).

The memory 330 is used to store application program codes for executing the solutions in the embodiments of the present disclosure, and the execution is performed by the processor 310. The processor 310 is used to execute the application program codes stored in the memory 330, so as to control the computer system 300 to implement the health data management method provided by the following embodiments of the present disclosure. Optionally, the computer-executed instructions in the embodiments of the present disclosure may also be referred to as application program codes, which is not limited in the embodiments of the present disclosure.

In an implementation, as an embodiment, the processor 310 may include one or more CPUs, such as CPU0 and CPU1 shown in FIG. 3A. Each CPU may support a plurality of virtual CPUs, which are also referred to as VCPUs.

In an implementation, as an embodiment, the computer system 300 may include a plurality of processors, such as the processor 310 and a processor 370 shown in FIG. 3A. Each of these processors may be a single-CPU processor or a multi-CPU processor. The processor here may be referred to as one or more devices, circuits, and/or processing cores for processing data (e.g. computer program instructions).

In an implementation, as an embodiment, the computer system 300 may further include an output device 350 and an input device 360. The output device 350 is in communication with the processor 310 and is able to display information in a variety of ways. For example, the output device 350 may be a liquid crystal display (LCD), a light-emitting diode (LED) display device, a cathode ray tube (CRT) display device, or a projector. The input device 360 is in communication with the processor 310 and is able to accept the user's input in a variety of ways. For example, the input device 360 may be a mouse, a keyboard, a touch screen device or a sensor device.

The computer system 300 may be a general communication device or a special communication device. The type of the computer system 300 is not limited in the embodiments of the present disclosure. In an implementation, the computer system 300 may be a desktop computer, a portable computer, a network server, a personal digital assistant (PDA), a mobile phone, a tablet computer, a wireless terminal device, an embedded device, or a device with a similar structure as a structure shown in FIG. 3. The various components in the computer system 300 may be simultaneously deployed in a same computer device, or may be deployed in different computer devices located in a distributed system.

Moreover, the terminal 103 shown in FIG. 1A may be a mobile phone, a tablet computer, a wearable device, an on-board device, an augmented reality (AR)/virtual reality (VR) device, a laptop, an ultra-mobile personal computer (UMPC), a netbook, a personal digital assistant (PDA), etc., and a type of the terminal 103 is not limited in the embodiments of the present disclosure.

The health data collection device 104 shown in FIG. 1A may be a sleep instrument, a sports watch, a wristband, a sphygmomanometer, a weight scale, a blood glucose meter, etc., and a type of the health data collection device 104 is not limited in the embodiments of the present disclosure.

For ease of understanding, considering an example in which the health data management system is a system having the structure shown in FIG. 1A, the health data management method provided by the embodiments of the present disclosure will be described with reference to the accompanying drawings.

Referring to FIG. 3B, the health data management method may include following steps 301 to 304.

In step 301, a first background device sends first health data to the health data management device. The first health data includes: a first user identifier, a first health index identifier, and a first data content corresponding to the first health index identifier.

The first background device may be any one of the background devices shown in FIG. 1A.

User identifiers are used to identify different users. That is, the user identifiers corresponding to the different users are different. The first user identifier is used to represent information of a first user. For example, the first user identifier may be one or more of the first user's identity (ID) card numbers, mobile phone numbers, a WeChat ID, a registered user name, and the like.

Health index identifiers are used to represent different types of health data contents. Different types of health data contents correspond to different health index identifiers. For example, the health index identifier of diastolic pressure may be DE04.10.176.00, the health index identifier of systolic pressure may be DE04.10.174.00, the health index identifier of glycated hemoglobin may be DE04.50.083.00, and the health index identifier of sleep may be DE04.60.063.00.

Data contents of health data may be divided into different types. For example, the types of data contents may be classified into three types: number, text, and option. In a case where a type of the data content is “number”, the data content may include a value range type and a value range. The value range type may be divided into range values, enumeration, a single value, etc., and the value range specifies a range in which the data can be input. In addition, in a case where the type of the data content is “number”, the data content may further include a reference value type and a reference value. The reference value type may also be divided into the range value, enumeration, the single value, etc., and the reference value specifies a normal range value of the data. In a case where the type of the data content is “text”, for example, the data content may be a diagnosis report or a treatment plan. In a case where the type of the data content is “option”, for example, the data content may be preset options. For example, the data content of the health data recording the user's eye vision condition may be one of the following options: A: myopia; B: hyperopia; C: normal.

In addition, in some implementations, the first background device may send the encrypted first health data to the health data management device. For example, the first background device may encrypt the first health data by using an advanced encryption standard (AES) and send the encrypted first health data to the health data management device. Of course, the first background device may also encrypt the first health data for encryption transmission through other encryption methods, and the encryption method is not limited in the embodiments of the present disclosure.

In step 302, the health data management device stores the first health data after receiving the first health data from the first background device.

In step 303, a second background device sends second health data to the health data management device.

The second background device may be any one of the background devices shown in FIG. 1A.

The second health data includes: a second user identifier, a second health index identifier, and a second data content corresponding to the second health index identifier. Data formats of the first health data and the second health data are the same. For example, the second user identifier may be one or more of a second user's identity (ID) card number, mobile phone numbers, a WeChat ID, a registered user name, and the like.

In a case where health types of the first data content and the second data content are the same, the first health index identifier and the second health index identifier are the same. In a case where health types of the first data content and the second data content are different, the first health index identifier and the second health index identifier are different. Therefore, the health types of the data contents are different, and the corresponding health index identifiers are also different. That is, the health types of the data contents and the health index identifiers have a corresponding relationship therebetween.

The data formats of the first health data and the second health data are the same. That is, the first health data and the second health data have a unified data format. For example, the unified data format is prescribed by the health data management device 101. For example, the unified data format may be a json format. For another example, the unified data format may be an XML format. For yet another example, the unified data format may be a CSV format. Of course, the unified data format may also be data formats of other types, and a type of the unified data format is not limited in the embodiments of the present disclosure.

For example, in a case where the unified data format is the json format, an example of the health data can be as follows:

```
{
“userId”: 123456,
“sys”: 1,
“gatherType”: 1,
“deviceType”: “EQ0001”,
“deviceId”: “QWERTYUIOP”,
“dataList”: [
  {
    “time”: “2019-07-02 14:09:00”,
    “data”[
      {
        “code”: “IND000009”,
        “value”: “a”,
        “valueType”: 1,
        “dataType”: 0,
      }
      {
        “code”: “IND000009”,
        “value”: “b”,
        “valueType”: 1,
        “dataType”: 0,
      }
      ]
  }
  ]
  }
```

Where “userId” represents a user's identifier; “sys” represents a system code; “gatherType” represents an input method, “0” indicates that health data is entered manually, and “1” indicates that health data is collected by the health data collection device; “deviceType” represents a device type (i.e., indicating what kind of device the device is); “deviceId” represents a unique identification code of a device (e.g., a MAC address); “dataList” represents a health data set; “time” represents measurement time of health data; “data” represents a specific data item; “code” represents an index identifier (also referred to as an index code); “value” represents a value of an index; “valueType” represents a type of a value, “0” represents a specific value, “1” represents a reference; “dataType” represents a data type, “0” represents a text, “1” represents an image, “2” represents a video, “3” represents an audio, and “4” represents an external link.

The health data may include at least one of the above fields, and the fields included in the health data sent by different background servers may be partially the same, or may be entirely the same.

In addition, in some implementations, the second background device may send the encrypted second health data to the health data management device, and the encryption method is not limited in the embodiments of the present disclosure.

In step 304, the health data management device stores the second health data after receiving the second health data from the second background device.

The steps 301 to 304 exemplarily illustrate the health data management method provided by the embodiments of the present disclosure, and do not constitute a limitation on the execution sequence of the method. For example, during an implementation, the method may be executed in a sequence from the step 301 to the step 304. Alternatively, the step 303 and the step 304 may also be executed first, and then the step 301 and the step 302 may be executed. Alternatively, the step 301 and the step 303 may also be executed first, and then the step 302 and the step 304 may be executed, wherein the step 301 and the step 303 may be executed simultaneously, and the step 302 and the step 304 may also be executed simultaneously. The execution sequence of the steps 301 to 304 is not limited in the embodiments of the present disclosure.

In the solution described in the steps 301 to 304, the health data management device may receive and store the first health data sent by the first background device and the second health data sent by the second background device. The data formats of the first health data and the second health data are the same. In the case where the health types of the first data content and the second data content are the same, the first health index identifier and the second health index identifier are the same. In the case where the health types of the first data content and the second data content are different, the first health index identifier and the second health index identifier are different. That is to say, the health data management device adopts a unified data format to store the health data, and the health data of the same health types have the same health index identifier. Therefore, the health data management device can aggregate health data from different background devices for analysis and processing, which may improve the accuracy and the reliability of processing the health data and thus improve user experience.

In addition, the unified management of data, compared with a solution that various background devices manage health data separately in the related art, may reduce the workload of development significantly, improve the efficiency of development, and reduce the labor costs.

In some implementations, in the above step 302 and step 304, the health data management device may store the first health data and the second health data by using TIDB. TIDB is an open source distributed newSQL database, which is able to realize automatic horizontal scaling and distributed transactions with strong consistency, and is compatible with the mysql protocol. Thus, it is easy to develop and has high query efficiency, and is suitable for storing a large amount of health data in the health data management devices.

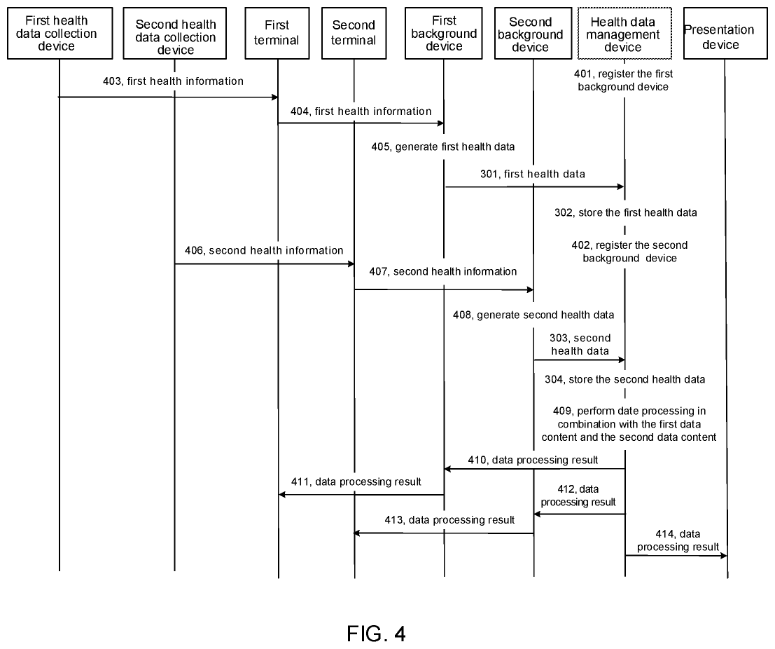

Optionally, in some embodiments, referring to FIG. 4, before the step S301, the method further includes a following step 401.

In step 401, the health data management device registers the first background device according to a received first registration request, and returns first information to the first background device.

The first information may include an identifier of the first background device and a key of the first background device.

That is to say, before the first background device sends the first health data to the health data management device, the health data management device registers the first background device according to the first registration request, and returns the first information to the first background device. As a result, before data transmission is performed between the first background device and the health data management device, the health data management device can authenticate the legitimacy of the first background device according to the first information, so as to ensure the security of data transmission. In addition, the registration of the first background device by the health data management device may also facilitate the management of the first background device by the health data management device. For example, the health data management device can disable the first background device.

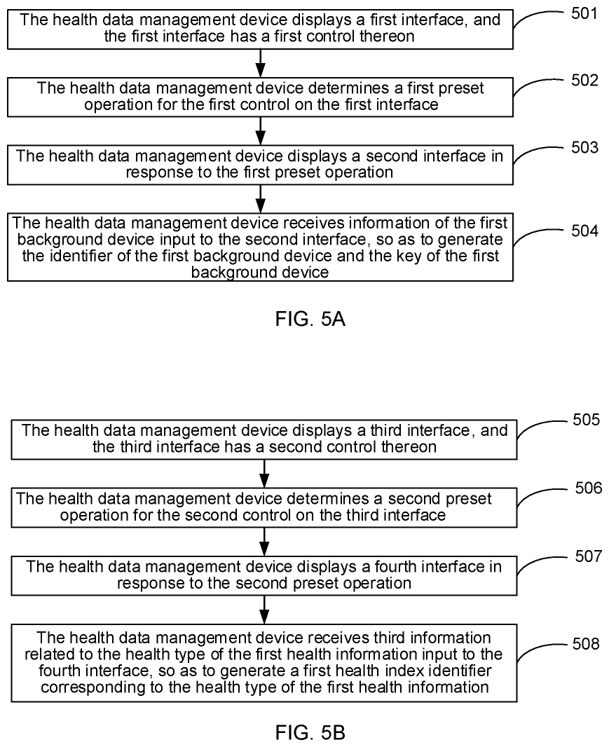

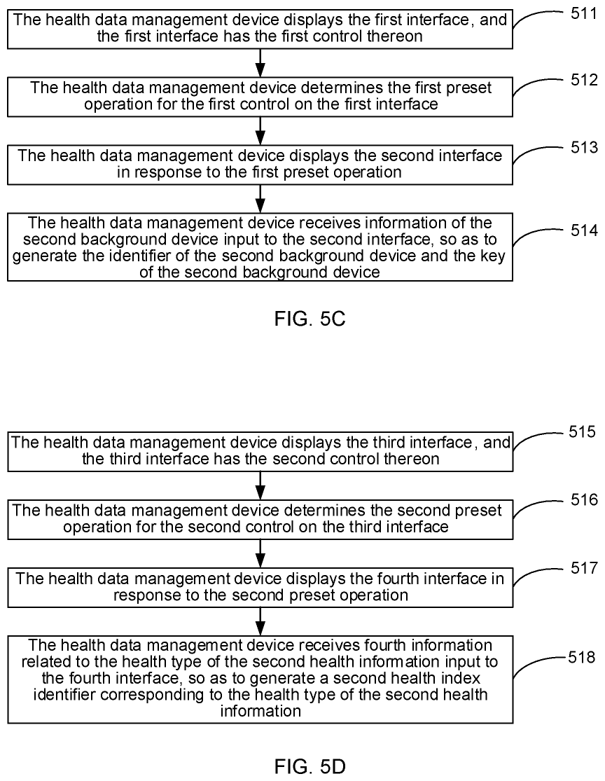

In some implementations, referring to FIG. 5A, the health data management device registers the first background device according to the received first registration request, which may include following steps 501 to 504.

In step 501, the health data management device displays a first interface, and the first interface has a first control thereon.

For example, the first interface may be the “External system” interface shown in FIG. 2B. The first control may be the control of “Create a new external system” 114 shown in FIG. 2B.

In step 502, the health data management device determines a first preset operation for the first control on the first interface.

For example, the first preset operation may be an operation of a single-click, a double-click, or a long press on the first control. Of course, the first preset operation may be other operations for the first control, and is not limited in the embodiments of the present disclosure.

In step 503, the health data management device displays a second interface in response to the first preset operation.

For example, the second interface may be the “Create a new external system” interface shown in FIG. 2C.

In step 504, the health data management device receives information of the first background device input to the second interface, so as to generate the identifier of the first background device and the key of the first background device.

For example, the input information of the first background device may be input specific information, shown in FIG. 2C, of the “System code”, the “System name”, the “Current version number” and the “System introduction”. Of course, the information of the first background device may also include more or less information, and the type of the information of the first background device is not limited in the embodiments of the present disclosure.

Therefore, the first registration request may include the first preset operation for the first control on the first interface and the information of the first background device input to the second interface.

That is to say, the health data management device can display the second interface based on the first preset operation for the first control on the first interface, and receive the information of the first background device input to the second interface, so as to generate the identifier of the first background device and the key of the first background device.

For example, the information of the first background device input to the second interface may come from an email sent by an administrator of the first background device to the administrator of the health data management device. The content of the email may include information such as the type of the first background device, the type of at least one first health data collection device for which the first background device provides service, and the health index identifier included in each first health data collection device. Therefore, the administrator can input the information of the first background device on the second interface according to the content of the email, so that the health data management device can complete the registration of the first background device, and registration may also be called creation. Of course, the information of the first background device input to the second interface may also have other forms, and the form of the information of the first background device input to the second interface is not limited in the embodiments of the present disclosure.

The first registration information obtained through the health data management device registering the first background device may include the identifier of the first background device and the key of the first background device. In addition, the first registration information may further include information such as the name of the first background device and the code of the first background device. The content of the first registration information is not limited in the embodiments of the present disclosure.

In some other implementations, referring to FIG. 5B, if the health index identifier corresponding to a health type of first health information is not provided, registering the first background device according to the received first registration request further includes: generating the first health index identifier corresponding to the health type of the first health information according to the received first registration request. The health type of the first health information is a health type of health information that is able to be collected by a health data collection device for which the first background device provides service.

In some implementations, generating the first health index identifier corresponding to the health type of the first health information according to the received first registration request may include following steps 505 to 508.

In step 505, the health data management device displays a third interface, and the third interface has a second control thereon.

For example, the third interface may be the “Index management” interface shown in FIG. 2G, and the second control may be the control of “Create an index” 117 in the “Index management” interface shown in FIG. 2G.

In step 506, the health data management device determines a second preset operation for the second control on the third interface.

For example, the second preset operation may be an operation of a single-click, a double-click, or a long press on the second control. Of course, the second preset operation may be other operations for the second control, and is not limited in the embodiments of the present disclosure.

In step 507, the health data management device displays a fourth interface in response to the second preset operation.

For example, the fourth interface may be the “Create an index” interface shown in FIG. 2H.

In step 508, the health data management device receives third information related to the health type of the first health information input to the fourth interface, so as to generate a first health index identifier corresponding to the health type of the first health information.

For example, the input third information related to the health type of the first health information may be specific information, shown in FIG. 2H, of the “Index name”, the “Index unit” and the “Index code”. Of course, the input third information related to the health type of the first health information may also include more or less information, and the type of the input third information related to the health type of the first health information is not limited in the embodiments of the present disclosure.

That is to say, the health data management device may display the fourth interface based on the second preset operation for the second control on the third interface, and receive the third information related to the health type of the first health information input to the fourth interface, so as to generate the first health index identifier corresponding to the health type of the first health information.

Optionally, in some other embodiments, referring to FIG. 4, before the step 303, the method further includes a following step 402.

In step 402, the health data management device registers the second background device according to a second registration request, and returns the second information to the second background device.

The second information may include an identifier of the second background device and a key of the second background device.

That is to say, before the second background device sends the second health data to the health data management device, the health data management device registers the second background device according to the second registration request, and returns the second information to the second background device. As a result, before data transmission is performed between the second background device and the health data management device, the health data management device can authenticate the legitimacy of the second background device according to the second information, so as to ensure the security of data transmission. In addition, the registration of the second background device by the health data management device also facilitates the management of the second background device by the health data management device. For example, the health data management device may disable the second background device.

In some implementations, referring to FIG. 5C, the health data management device registering the second background device according to the received second registration request may include following steps 511 to 514.

In step 511, the health data management device displays the first interface, and the first interface has the first control thereon.

For example, the first interface may be the “External system” interface shown in FIG. 2B. The first control may be the control of “Create a new external system” 114 shown in FIG. 2B. The step 511 may be the same as the step 501.

In step 512, the health data management device determines the first preset operation for the first control on the first interface.

For example, the first preset operation may be an operation of a single-click, a double-click, or a long press on the first control. Of course, the first preset operation may be other operations for the first control, and is not limited in the embodiments of the present disclosure. The step 512 may be the same as the step 502.

In step 513, the health data management device displays the second interface in response to the first preset operation.

For example, the second interface may be the “Create a new external system” interface shown in FIG. 2C. The step 513 may be the same as the step 503.

In step 514, the health data management device receives information of the second background device input to the second interface, so as to generate the identifier of the second background device and the key of the second background device.

For example, the input information of the second background device may be input specific information, shown in FIG. 2C, of the “System code”, the “System name”, the “Current version number” and the “System introduction”. Of course, the information of the second background device may also include more or less information, and the type of the information of the second background device is not limited in the embodiments of the present disclosure.

Therefore, the second registration request may include the first preset operation for the first control on the first interface and the information of the second background device input to the second interface.

That is to say, the health data management device can display the second interface based on the first preset operation for the first control on the first interface, and receive the information of the second background device input to the second interface, so as to generate the identifier of the second background device and the key of the second background device.

For example, the information of the second background device input to the second interface may come from an email sent by an administrator of the second background device to the administrator of the health data management device. The content of the email may include information such as the type of the second background device, the type of at least one second health data collection device for which the second background device provides service, and the health index identifier included in each second health data collection device. Therefore, the administrator can input the information of the second background device to the second interface according to the content of the email, so that the health data management device can complete the registration of the second background device. Of course, the information of the second background device input to the second interface may also have other forms, and the form of the information of the second background device input to the second interface is not limited in the embodiments of the present disclosure.

The second registration information obtained through the health data management device registering the second background device may include the identifier of the second background device and the key of the second background device. In addition, the second registration information may further include information such as the name of the second background device and the code of the second background device. The content of the second registration information is not limited in the embodiments of the present disclosure.

In some other implementations, referring to FIG. 5D, if the health index identifier corresponding to a health type of second health information is not provided, registering the second background device according to the received second registration request further includes: generating the second health index identifier corresponding to the health type of the second health information according to the received second registration request. The health type of the second health information is a health type of the health information that is able to be collected by a health data collection device for which the second background device provides service.

In some implementations, generating the second health index identifier corresponding to the health type of the second health information according to the received second registration request may include following steps 515 to 518.

In step 515, the health data management device displays the third interface, and the third interface has the second control thereon.

In step 516, the health data management device determines the second preset operation for the second control on the third interface.

In step 517, the health data management device displays the fourth interface in response to the second preset operation.

The steps 515 to 517 may be the same as the steps 505 to 507 respectively, and will not be repeated herein.

In step 518, the health data management device receives fourth information related to the health type of the second health information input to the fourth interface, so as to generate a second health index identifier corresponding to the health type of the second health information.

For example, the input fourth information related to the health type of the second health information may be specific information, shown in FIG. 2H, of the “Index name”, the “Index unit” and the “Index code”. Of course, the input fourth information related to the health type of the second health information may also include more or less information, and the type of the input fourth information related to the health type of the second health information is not limited in the embodiments of the present disclosure.

That is to say, the health data management device may display the fourth interface based on the second preset operation of the second control on the third interface, and receive the fourth information related to the health type of the second health information input to the fourth interface, so as to generate the second health index identifier corresponding to the health type of the second health information.

In addition, optionally, in some implementations, referring to FIG. 4, after the step 401 and before the step 301, the method may further include following steps 403 to 405.

In step 403, a first health data collection device sends collected first health information to a first terminal.

The first health information may include first user information and the first data content. The first user information is information used to indicate the first user. Different users may correspond to different user information.

The first terminal is connected to a first background device. Each background device has at least one corresponding terminal, and the terminal can be used to receive and display the health data and processing result that are from the background device.

In step 404, the first terminal sends the first health information to a first background device after receiving the first health information.

In step 405, the first background device generates the first health data based on the first health information, a configured first health index identifier corresponding to the health type of the first data content, and the fourth information after receiving the first health information.

The fourth information is used to indicate the data format of the health data. The fourth information may be configured information used to indicate the data format of the health data based on the information from the health data management device.

Information for indicating the data format of the health data sent by the health data management device to different background devices may be the same. That is, the health data management device may specify a unified data format.

The first health data may include: the first user identifier, the first data content and the first health index identifier. The first user identifier is obtained based on the first user information in the first health information. The first user information and the first user identifier are used to represent the same user. The first health index identifier is the received health index identifier corresponding to the health type of the first data content.

That is to say, the first health data collection device may send the collected first health information to the first terminal, and then the first terminal transmits the first health information to the first background device. After receiving the first health information, the first background device can generate the first health data with a unified data format based on the first health information, the configured first health index identifier corresponding to the health type of the first data content, and the fourth information.

Thus, the first health data is uploaded to the health data management device, and the health data management device performs unified storage and unified processing.

In addition, optionally, in some implementations, referring to FIG. 4, after the step 402 and before the step 303, the method may further include following steps 406 to 408.

In step 406, a second health data collection device sends collected second health information to a second terminal.

The second health information may include second user information and the second data content. The second user information is information used to indicate the second user. Different users may correspond to different user information. The second terminal is connected to a second background device.

In step 407, the second terminal sends the second health information to a second background device after receiving the second health information.

In step 408, the second background device generates the second health data based on the second health information (including the second user information and the second data content), the fourth information, and a configured second health index identifier corresponding to the health type of the second data content (in other words, a corresponding relationship between the health type of the data content and the health index identifier) after receiving the second health information.

The second background device parses and packs the second health information based on the second user information and the second data content in the second health information, and the corresponding relationship between the health type of the data content and the health index identifier, so as to generate the second health data with a unified data format.

That is to say, the second health data collection device may send the collected second health information to the second terminal, and then the second terminal transmits the second health information to the second background device. After receiving the second health information, the second background device can generate the second health data with a unified data format based on the second health information and the corresponding relationship between the health type of the data content and the health index identifier. Thus, the second health data is uploaded to the health data management device, and the health data management device performs unified storage and unified processing.

Optionally, in some other embodiments, referring to FIG. 4, after the step 304, the health data management method provided by the embodiments of the present disclosure further includes a following step 409.

In step 409, in a case where the first health index identifier and the second health index identifier are the same, the health data management device performs data processing in combination with the first data content and the second data content, so as to obtain a data processing result.

It will be noted from the above that, the first health index identifier and the second health index identifier are the same, so that the health type of the first data content of the first health data and the health type of the second data content of the second health data are the same. That is to say, in the case where the first health index identifier and the second health index identifier are the same, the health data management device can perform the data processing in combination with the first data content and the second data content, which may help improve the accuracy and the reliability of the processing result.

Optionally, in some implementations, the step 409 may include the following contents.

For example, in a case where data types of the first data content and the second data content are both numbers, weighted average processing is performed on the first data content and the second data content to obtain the data processing result.

For example, in a case where the data types of the first data content and the second data content are both texts, a part, having the same semantics in the first data content and the second data content, of the first data content or the second data content is deleted.

The description that the part, having the same semantics in the first data content and the second data content, of the first data content or the second data content is deleted means that, for the part with the same semantics in the first data content and the second data content, only one part is reserved.

The data type of the data content may be a text, a picture, a video, an audio, an external link, etc., and is not limited in the embodiments of the present disclosure. Of course, in the case where the data types of the first data content and the second data content are both numbers or both texts, the implementation of the step 409 is not limited to the above example, and the implementation of the step 409 is not limited in the embodiments of the present disclosure.

Optionally, in another embodiment, after the step 409, the method further includes following steps 410 and 411.

In step 410, the health data management device transmits the data processing result to the first background device.

In step 411, the first background device transmits the data processing result to the first terminal after receiving the data processing result.

Optionally, in another embodiment, the method may further include following steps 412 and 413.

In step 412, the health data management device transmits the data processing result to the second background device.

In step 413, the second background device transmits the data processing result to the second terminal after receiving the data processing result.

That is to say, after the health data management device performs the data processing in combination with the first data content and the second data content and obtains the data processing result, the health data management device may transmit the data processing result to the first background device or the second background device. As a result, the first background device may display the data processing result to the user through the first terminal connected thereto, or the second background device may display the data processing result to the user through the second terminal connected thereto.

In some other embodiments, the method further includes a following step 414.

In step 414, the health data management device transmits the data processing result to the presentation device.

For example, the health data management device may transmit the data processing result to the presentation device other than the first background device and the second background device through an external interface.

That is to say, after the health data management device performs the data processing in combination with the first data content and the second data content and obtains the data processing result, the health data management device may transmit the data processing result to the presentation device other than the first background device and the second background device, so that the data processing result can be displayed to the user through the presentation device.

In another embodiment, the method may further include: displaying the data processing result by the health data management device.

That is to say, the health data management device can also directly provide a display page itself, so that the user can view the data processing result through the display page.

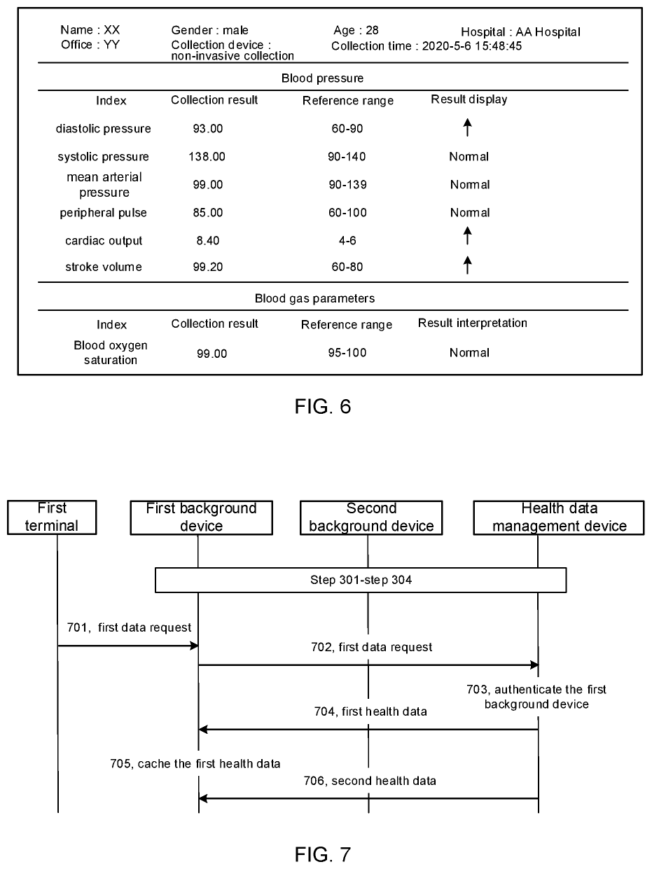

In some implementations, the health data management device may provide the display page for some authorized users, so that these users can view the data processing result. For example, the authorized users may be staff of the first background device and/or the second background device. For example, in a case where the background device is a patient information management server of a certain hospital, the authorized users may be doctors working in the hospital. For example, referring to FIG. 6, it shows a schematic diagram of the display page provided by the health data management device.

Optionally, in some embodiments, referring to FIG. 7, after the step 304, the method may further include following steps 701 to 704.

In step 701, the first terminal sends a first data request to the first background device in response to a preset operation of a first terminal user.

The preset operation of the first terminal user may be used to instruct to view health data of the first user. For example, the preset operation of the first terminal user may be an operation that the first terminal user clicks a preset control on a terminal screen. For example, the preset operation of the first terminal user may be an operation that the first terminal user clicks a “Health data query” button on the terminal screen. For another example, the preset operation of the first terminal user may be a preset gesture operation of the first terminal user on the terminal screen. For example, the preset operation of the first terminal user may be a gesture operation of drawing a circle on the terminal screen. Of course, the preset operation of the first terminal user may also have other forms, which are not limited in the embodiments of the present disclosure.

The first data request is used to request the health data of the first user, and the first data request includes the identifier of the first background device and the key of the first background device.

For example, in a case where the first background device is a mobile health server, the first terminal user may be a user of a wristband for which the mobile health server provides service, and the first data request may be used to request the health data of the user of the wristband.

In some embodiments, the first data request may also be used to request health data of users in addition to the first user. That is, the first data request may be used to request health data of multiple users including the first user.

For example, in a case where the background device is the patient information management server of the certain hospital, the first terminal user may be a doctor working in the hospital, and the first data request may be used to request health data of multiple patients managed by the doctor.

In step 702, after receiving the first data request from the first terminal, the first background device sends the first data request to the health data management device.

In step 703, the health data management device authenticates the first background device in response to the first data request.

The health data management device may authenticate the first background device in response to both the identifier of the first background device and the key of the first background device included in the first data request.

In step 704, the health data management device transmits the first health data to the first background device if the authentication of the first background device by the health data management device passes.

That is to say, when the first background device requests the health data of the first user from the health data management device, the health data management device may authenticate the legitimacy of the first background device according to both the identifier of the first background device and the key of the first background device included in the first data request, and transmit the first health data after the authentication passes, so as to ensure the security of data transmission.

Optionally, after the step 704, the method may further include a following step 705.

In step 705, after receiving the first health data, the first background device caches the first health data.

For example, the first health data may be cached in a queue. For example, the queue may be a first-in, first-out queue.

Therefore, in a case where the first background device receives the first data request from the first terminal again, if the first health data is still cached in the first background device, the first background device may directly send the first health data to the first terminal without a further request from the health management server, so as to save transmission resources and time.

In another embodiment, after the step 705, the method further includes a following step 706.

In step 706, in a case where the first user identifier and the second user identifier are the same, if the authentication of the first background device by the health data management device passes, the health data management device further transmits the second health data to the first background device.

That is to say, if the first user and the second user are the same user, in a case where the authentication of the first background device by the health data management device passes, the health data management device not only transmits the first health data from the first background device to the first background device, but also transmits the second health data from the second background device to the first background device. That is, in addition to the health data uploaded by the background device itself, the background device may further obtain health data uploaded by other background devices from the health data management device, so that health data from different background devices may interconnect, which may improve the flexibility and the efficiency of the use of these data, reduce the development costs and improve the development efficiency.

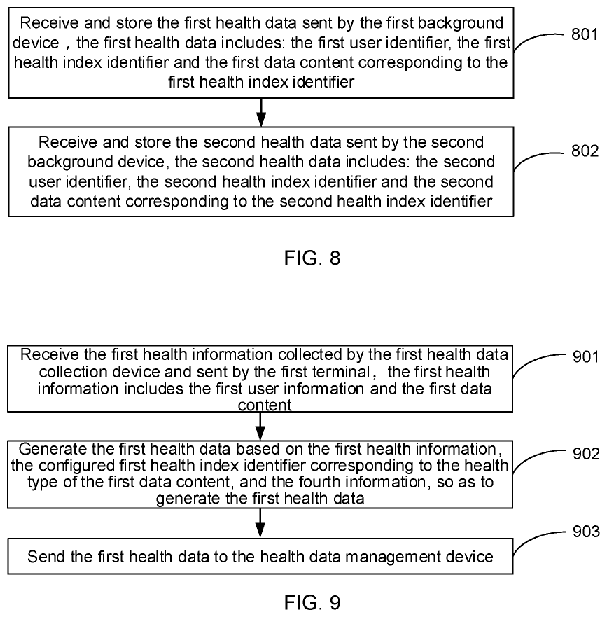

In summary, the embodiments of the present disclosure provide a health data management method, which is applied to the health data management device. In an embodiment, as shown in FIG. 8, the method may include following steps 801 to 802.

In step 801, the first health data sent by the first background device is received and stored. The first health data includes: the first user identifier, the first health index identifier and the first data content corresponding to the first health index identifier.

In step 802, the second health data sent by the second background device is received and stored. The second health data includes: the second user identifier, the second health index identifier and the second data content corresponding to the second health index identifier.

Data formats of the first health data and the second health data are the same. In a case where the health types of the first data content and the second data content are the same, the first health index identifier and the second health index identifier are the same. In a case where the health types of the first data content and the second data content are different, the first health index identifier and the second health index identifier are different.

In the above health data management method, for example, the operation of the health data management device may be referred to the operation of the health data management device in the embodiment shown in FIG. 3 and above relevant description, which will not be described in detail here.

In addition, the embodiments of the present disclosure further provide another health data management method, which is applied to the first background device. As shown in FIG. 9, the method includes the following steps 901 to 903.

In step 901, the first health information collected by the first health data collection device and sent by the first terminal is received. The first health information includes the first user information and the first data content.

In step 902, the first health data is generated based on the first health information, the configured first health index identifier corresponding to the health type of the first data content, and the fourth information, so as to generate the first health data.

The fourth information is used to indicate the data format of the health data, the health data may include various types of health data, and the health data includes the first health data. The first health data includes the first user identifier, the first data content and the first health index identifier. The first user information and the first user identifier are used to represent the same user.

In step 903, the first health data is sent to the health data management device.

In the above health data management method, for example, the operation of the first background device may be referred to the operation of the first background device in the embodiment shown in FIG. 4 and above relevant description, which will not be described in detail here.

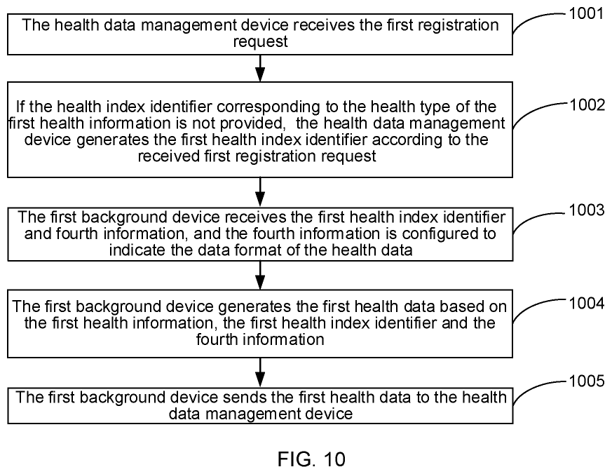

In addition, the embodiments of the present disclosure further provide another health data management method. Referring to FIG. 10, the method includes following steps1001 to 1005.

In step 1001, the health data management device receives the first registration request.

The first registration request is configured to instruct the registration of the first background device.

In step 1002, if the health index identifier corresponding to the health type of the first health information is not provided, the health data management device generates the first health index identifier according to the received first registration request.

The first health index identifier is configured to identify the health type of the first health information. The health type of the first health information is the health type of the health information that is able to be collected by the health data collection device for which the first background device provides service.

In step 1003, the first background device receives the first health index identifier and fourth information, and the fourth information is configured to indicate the data format of the health data.

In step 1004, the first background device generates the first health data based on the first health information, the first health index identifier and the fourth information.

The first health data includes the first health index identifier. The first health index identifier is used to identify the health type of the first health data (or, the first health information).

In step 1005, the first background device sends the first health data to the health data management device.

In the above health data management method, for example, the operations of both the health data management device and the first background device may be referred to the operations of both the health data management device and the first background device in the embodiments shown in FIGS. 4 and 7 and above relevant description, which will not be repeated here.

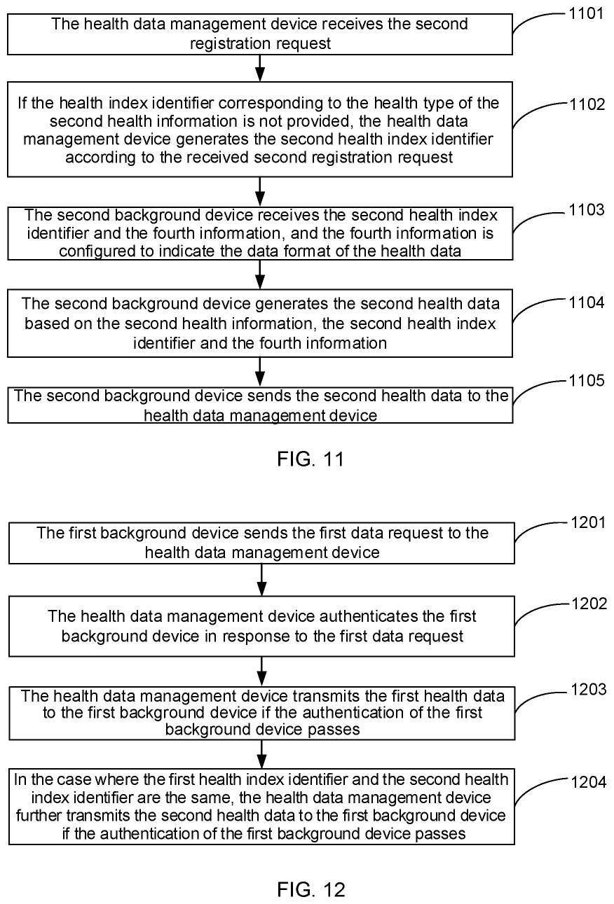

In some embodiments, referring to FIG. 11, the method may further include following steps 1101 to 1105.

In step 1101, the health data management device receives the second registration request.

The second registration request is configured to instruct the registration of the second background device.

In step 1102, if the health index identifier corresponding to the health type of the second health information is not provided, the health data management device generates the second health index identifier according to the received second registration request.

The second health index identifier is configured to identify the health type of the second health information. The health type of the second health information is the health type of the health information that is able to be collected by the health data collection device for which the second background device provides service.

In step 1103, the second background device receives the second health index identifier and the fourth information, and the fourth information is configured to indicate the data format of the health data.

In step 1104, the second background device generates the second health data based on the second health information, the second health index identifier and the fourth information.

The second health data includes the second health index identifier.

In step 1105, the second background device sends the second health data to the health data management device.

In the above health data management method, for example, the operations of both the health data management device and the second background device may be referred to the operations of both the health data management device and the second background device in the embodiments shown in FIGS. 4 and 7 and above relevant description, which will not be repeated here.

In some other embodiments, referring to FIG. 12, the method may further include following steps 1201 to 1203.

In step 1201, the first background device sends the first data request to the health data management device.

The first data request is configured to request the health data of the first user, and the first data request includes the identifier of the first background device and the key of the first background device.

In step 1202, the health data management device authenticates the first background device in response to the first data request.

In step 1203, the health data management device transmits the first health data to the first background device if the authentication of the first background device passes.

In addition, in a case where the first health index identifier and the second health index identifier are the same, after the step 1203, the method may further include a following step 1204.

In step 1204, in the case where the first health index identifier and the second health index identifier are the same, the health data management device further transmits the second health data to the first background device if the authentication of the first background device passes.

That is to say, in the case where the first health index identifier and the second health index identifier are the same, in addition to obtaining the first health data uploaded by the first background device itself from the health data management device, the first background device can further obtain the second health data uploaded by the second background device, so that the health data from different background devices may interconnect, which may improve the flexibility and the efficiency of the use of these data, reduce the development costs and improve the development efficiency.

The health data management device, the background devices, the terminals, and the health data collection devices may each be referred to as an electronic device. It will be understood that, in order to achieve the above functions, the electronic device includes corresponding hardware and/or software for performing various functions. In conjunction with algorithm steps of examples described in the embodiments disclosed herein, the present disclosure can be implemented in a form of hardware or in a form of a combination of hardware and computer software. Whether a certain function is performed through hardware or through computer software driving hardware depends on the specific application of the technical solution and restrictive conditions on design. A person skilled in the art may implement the described function of each specific application by using different methods in combination with the embodiments, but such implementation should not be considered beyond the scope of the present disclosure.

Functional modules of the electronic device may be divided based on the above examples of methods in the embodiments. For example, each function module may be divided according to its function, two or more functions may also be integrated into one processing module. The integrated module may be implemented in a form of hardware. It will be noted that, a division of the module in some embodiments is schematic, and it is only a logical functional division, and there may be other ways to divide the module in actual implementation.

In a case where each functional module is divided according to its function, the health data management device provided by the embodiments of the present disclosure may be shown as FIG. 1B. Referring to FIG. 1B, the health data management device 101 may include the storage unit 110 and the transceiver unit 120.

The transceiver unit 120 is used to receive the first health data sent by the first background device. The first health data includes the first user identifier, the first health index identifier, and the first data content corresponding to the first health index identifier.

The transceiver unit 120 is further used to receive the second health data sent by the second background device. The second health data includes the second user identifier, the second health index identifier, and the second data content corresponding to the second health index identifier. The data formats of the first health data and the second health data are the same. In the case where the health types of the first data content and the second data content are the same, the first health index identifier and the second health index identifier are the same. In the case where the health types of the first data content and the second data content are different, the first health index identifier and the second health index identifier are different.

The storage unit 110 is used to store the first health data and store the second health data.

In some embodiments, the health data management device further includes the processing unit 130 and the processing unit 130 may include an external system management module 1011.

The external system management module 1011 may be used to: display the first interface, the first interface having the first control thereon; determine the first preset operation for the first control on the first interface; display the second interface in response to the first preset operation; and receive the information of the first background device input to the second interface, so as to generate the identifier of the first background device and the key of the first background device.

The external system management module 1011 may further be used to: display the first interface, the first interface having the first control thereon; determine the first preset operation for the first control on the first interface; display the second interface in response to the first preset operation; and receive the information of the second background device input to the second interface, so as to generate the identifier of the second background device and the key of the second background device.

In some other embodiments, the processing unit 130 further includes the index management module 1012. The index management module 1012 may be used to: display the third interface, the third interface having the second control thereon; determine the second preset operation for the second control on the third interface; display the fourth interface in response to the second preset operation; and receive the third information related to the health type of the first health information input to the fourth interface, so as to generate the first health index identifier corresponding to the health type of the first health information.

The index management module 1012 may also be used to: display the third interface, the third interface having the second control thereon; determine the second preset operation for the second control on the third interface; display the fourth interface in response to the second preset operation: and receive the fourth information related to the health type of the second health information input to the fourth interface, so as to generate the second health index identifier corresponding to the health type of the second health information.

In some other embodiments, the processing unit 130 may be used to, in a case where the first health index identifier and the second health index identifier are the same, and the first user identifier and the second user identifier are the same, perform data processing in combination with the first data content and the second data content, so as to obtain the data processing result.

In some other embodiments, the transceiver unit 120 further includes the unified data transmission interface 1013 and the external interface 1014.

The unified data transmission interface 1013 can be used to transmit the data processing result to the first background device. The unified data transmission interface 1013 can further be used to transmit the data processing result to the second background device.

The external interface 1014 can be used to transmit the data processing result to the presentation device.

In some other embodiments, the transceiver unit 120 may also be used to receive the first data request from the first background device. The first data request is configured to request the health data of the first user, and the first data request includes the identifier of the first background device and the key of the first background device.

The processing unit 130 may further be used to authenticate the first background device in response to the first data request; and transmit the first health data to the first background device if the authentication of the first background device passes.

In some other embodiments, the unified data transmission interface 1013 may also be used to, in the case where the first user identifier and the second user identifier are the same, transmit the second health data to the first background device if the authentication of the first background device passes.

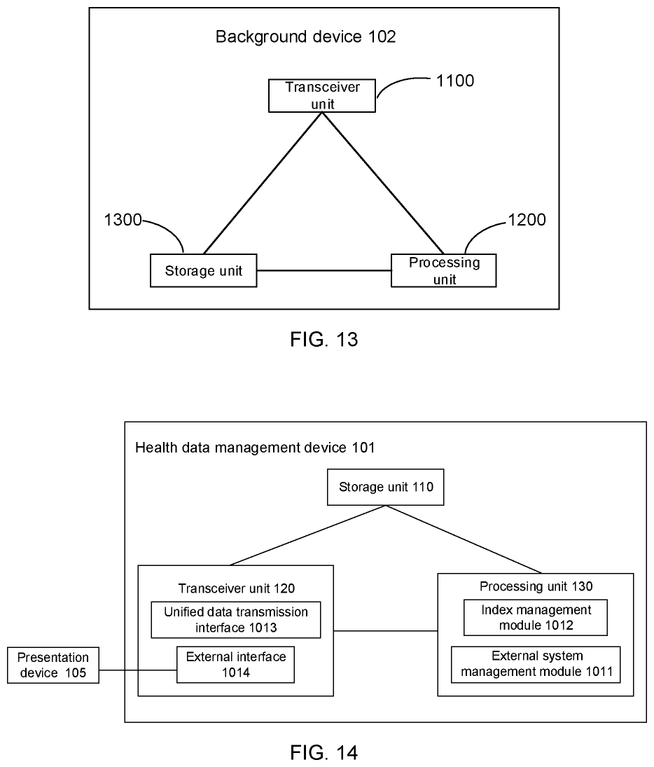

In addition, the background device provided by the embodiments of the present disclosure may be shown in FIG. 13. Referring to FIG. 13, the background device 102 may include a transceiver unit 1100 and a processing unit 1200.

The transceiver unit 1100 may be used to receive the first health information collected by the first health data collection device and sent by the first terminal, and the first health information includes the first user information and the first data content.

The processing unit 1200 may be used to generate the first health data based on the first health information, the configured first health index identifier corresponding to the health type of the first data content, and the fourth information. The fourth information is used to indicate the data format of the health data, the first health data includes the first user identifier, the first data content and the first health index identifier, and the first user information and the first user identifier are used to represent the same user.

The transceiver unit 1100 may also be used to send the first health data to the health data management device.

In some embodiments, the transceiver unit 1100 may further be used to: receive the first data request from the first terminal, the first data request being configured to request the health data of the first user, and the first data request including the identifier of the background device and the key of the background device; send the first data request to the health data management device; and receive the first health data from the health data management device.

In some other embodiments, in the case where the first user identifier is the same as the second user identifier of the second health data, the transceiver unit 1100 may further be used to receive the second health data from the health data management device. The second health data is the health data stored in the health data management device and from another background device other than the background device, and the second health data includes the second user identifier, the second health index identifier, and the second data content corresponding to the second health index identifier.

In some other embodiments, referring to FIG. 13, the background device 102 may further include a storage unit 1300. The storage unit 1300 may be used to cache the first health data.

Some embodiments of the present disclosure further provide a computer-readable storage medium (e.g., a non-transitory computer-readable storage medium). The computer-readable storage medium has stored thereon computer program instructions that, when run on a processor, cause the computer (e.g., the health data management device or the background device) to perform one or more steps of the health data management method as described in any one of the above embodiments.

For example, the computer-readable storage medium includes but is not limited to, a magnetic storage device (e.g., a hard disk, a floppy disk or a magnetic tape), an optical disk (e.g., a compact disk (CD), a digital versatile disk (DVD)), a smart card, a flash memory device (e.g., an erasable programmable read-only memory (EPROM)), a card, a stick or a key driver). Various computer-readable storage media described in the present disclosure may represent one or more devices and/or other machine-readable storage media, which are used for storing information. The term “machine-readable storage media” may include, but is not limited to, wireless channels and various other media capable of storing, containing and/or carrying instructions and/or data.

Some embodiments of the present disclosure further provide a computer program product. The computer program product includes computer program instructions that, when executed by a computer (e.g., the health data management device or the background device), cause the computer to perform one or more steps of the health data management method as described in the above embodiments.

Some embodiments of the present disclosure further provide a computer program. When the computer program is executed on the computer (e.g., the health data management device or the background device), the computer program causes the computer to perform one or more steps of the health data management method as described in the above embodiments.

Beneficial effects of the computer-readable storage medium, the computer program product and the computer program are the same as the beneficial effects of the health data management method as described in some of the above embodiments, which will not be described here again.

The foregoing descriptions are merely specific implementations of the present disclosure, but the protection scope of the present disclosure is not limited thereto. Changes or replacements that any person skilled in the art could conceive of within the technical scope of the present disclosure shall be included in the protection scope of the present disclosure. Therefore, the protection scope of the present disclosure shall be subject to the protection scope of the claims.

1. A health data management method, comprising:
receiving and storing first health data sent by a first background device, the first health data including a first health index identifier and a first data content corresponding to the first health index identifier; and
receiving and storing second health data sent by a second background device, the second health data including a second health index identifier and a second data content corresponding to the second health index identifier, wherein
data formats of the first health data and the second health data are same; if health types of the first data content and the second data content are same, the first health index identifier and the second health index identifier are same; if the health types of the first data content and the second data content are different, the first health index identifier and the second health index identifier are different.

2. The health data management method according to claim 1, wherein
before receiving and storing the first health data sent by the first background device, the method further comprises:
registering the first background device according to a received first registration request, and
returning first information to the first background device, wherein the first information includes an identifier of the first background device and a key of the first background device; and/or,
before receiving and storing the second health data sent by the second background device, the method further comprises:
registering the second background device according to a received second registration request, and
returning second information to the second background device, wherein the second information includes an identifier of the second background device and a key of the second background device.

3. The health data management method according to claim 2, wherein in a case where the health data management method includes registering the first background device according to the received first registration request,
if a health index identifier corresponding to a health type of first health information is not provided, registering the first background device according to the received first registration request includes:
generating the first health index identifier corresponding to the health type of the first health information according to the received first registration request, wherein the health type of the first health information is a health type of health information that is able to be collected by a health data collection device for which the first background device provides service;
in a case where the health data management method includes registering the second background device according to the received second registration request, if a health index identifier corresponding to a health type of second health information is not provided, registering the second background device according to the received second registration request includes:
generating the second health index identifier corresponding to the health type of the second health information according to the received second registration request, wherein the health type of the second health information is a health type of health information that is able to be collected by a health data collection device for which the second background device provides service.

4. The health data management method according to claim 1, wherein the first health index identifier and the second health index identifier are the same; after receiving and storing the first health data sent by the first background device, and receiving and storing the second health data sent by the second background device, the method further comprises:
performing data processing in combination with the first data content and the second data content to obtain a data processing result; and
transmitting the data processing result to at least one of the first background device, the second background device or a presentation device.

5. The health data management method according to claim 1, wherein after receiving and storing the first health data sent by the first background device, and receiving and storing the second health data sent by the second background device, the method further comprises:
receiving a first data request from the first background device, wherein the first data request is configured to request health data of a first user, the health data of the first user is the first health data, and the first data request includes an identifier of the first background device and a key of the first background device;
authenticating the first background device in response to the first data request; and
transmitting the first health data to the first background device if authentication of the first background device passes.

6. The health data management method according to claim 5, wherein the first health data further includes a first user identifier, the second health data further includes a second user identifier, and the first user identifier and the second user identifier are same; the method further comprises: transmitting the second health data to the first background device if the authentication of the first background device passes.

7. A health data management method, comprising:
receiving first health information collected by a first health data collection device and sent by a first terminal, wherein the first health information includes first user information and a first data content;
generating first health data based on the first health information, a configured first health index identifier corresponding to a health type of the first data content, and fourth information, wherein the fourth information is configured to indicate a data format of health data, the first health data includes a first user identifier, a first data content and a first health index identifier, and the first user information and the first user identifier are configured to represent a same user; and
sending the first health data to a health data management device.

8. The health data management method according to claim 7, wherein after sending the first health data to the health data management device, the method further comprises:
receiving a first data request from the first terminal, wherein the first data request is configured to request health data of a first user, the health data of the first user is the first health data, and the first data request includes an identifier of the first background device and a key of the first background device;
sending the first data request to the health data management device; and
receiving the first health data from the health data management device.

9. The health data management method according to claim 8, wherein the first user identifier is same as a second user identifier of second health data; after sending the first data request to the health data management device, the method further comprises:
receiving the second health data from the health data management device, wherein the second health data is health data from a second background device and stored in the health data management device, and the second health data includes the second user identifier, a second health index identifier and a second data content corresponding to the second health index identifier.

10. A health data management method, comprising:
receiving, by a health data management device, a first registration request, wherein the first registration request is configured to instruct to register a first background device;
generating, by the health data management device, a first health index identifier according to the received first registration request if a health index identifier corresponding to a health type of first health information is not provided, wherein the first health index identifier is configured to identify the health type of the first health information, and the health type of the first health information is a health type of health information that is able to be collected by a health data collection device for which the first background device provides service;
receiving, by the first background device, the first health index identifier and fourth information, wherein the fourth information is configured to indicate a data format of health data;
generating, by the first background device, first health data based on the first health information, the first health index identifier and the fourth information, wherein the first health data includes the first health index identifier; and
sending, by the first background device, the first health data to the health data management device.

11. The health data management method according to claim 10, further comprising:
receiving, by the health data management device, a second registration request, wherein the second registration request is configured to instruct to register a second background device;
generating, by the health data management device, a second health index identifier according to the received second registration request if a health index identifier corresponding to a health type of second health information is not provided, wherein the second health index identifier is configured to identify the health type of the second health information, and the health type of the second health information is a health type of health information that is able to be collected by a health data collection device for which the second background device provides service;
receiving, by the second background device, the second health index identifier and the fourth information, wherein the fourth information is configured to indicate the data format of the health data;
generating, by the second background device, second health data based on the second health information, the second health index identifier and the fourth information, wherein the second health data includes the second health index identifier; and
sending, by the second background device, the second health data to the health data management device.

12. The health data management method according to claim 11, further comprising:
sending, by the first background device, a first data request to the health data management device, wherein the first data request is configured to request health data of a first user, the health data of the first user is the first health data, and the first data request includes an identifier of the first background device and a key of the first background device;
authenticating, by the health data management device, the first background device in response to the first data request;
transmitting, by the health data management device, the first health data to the first background device if authentication of the first background device passes; and
transmitting, by the health data management device, the first health data and the second health data to the first background device if the authentication of the first background device passes and the first health index identifier and the second health index identifier are same.

13-17. (canceled)

18. A health data management device, comprising: a processor and a memory, wherein the memory is configured to store computer instructions that, when executed by the processor, cause the health data management device to perform the method according to claim 1.

19. (canceled)

20. A non-transitory computer-readable storage medium, wherein the computer-readable storage medium has stored thereon computer program instructions that, when run on a processor, cause the processor to perform the health data management method according to claim 1.

21. The health data management method according to claim 2, wherein in a case where the health data management method includes registering the first background device according to the received first registration request, registering the first background device according to the received first registration request includes:
displaying a first interface, the first interface having a first control thereon;
determining a first preset operation for the first control on the first interface;
displaying a second interface in response to the first preset operation; and
receiving information of the first background device input to the second interface, so as to generate the identifier of the first background device and the key of the first background device;
in a case where the health data management method includes registering the second background device according to the received second registration request, registering the second background device according to the received second registration request includes:
displaying the first interface, the first interface having the first control thereon;
determining the first preset operation for the first control on the first interface;
displaying the second interface in response to the first preset operation; and
receiving information of the second background device input to the second interface, so as to generate the identifier of the second background device and the key of the second background device.

22. The health data management method according to claim 3, wherein generating the first health index identifier corresponding to the health type of the first health information according to the received first registration request includes:
displaying a third interface, the third interface having a second control thereon;
determining a second preset operation for the second control on the third interface;
displaying a fourth interface in response to the second preset operation; and
receiving third information related to the health type of first health information input to the fourth interface, so as to generate the first health index identifier corresponding to the health type of the first health information; and
generating the second health index identifier corresponding to the health type of the second health information according to the received second registration request includes:
displaying the third interface, the third interface having the second control thereon;
determining the second preset operation for the second control on the third interface;
displaying the fourth interface in response to the second preset operation; and
receiving fourth information related to the health type of second health information input to the fourth interface, so as to generate the second health index identifier corresponding to the health type of the second health information.

23. A background device, comprising: a processor and a memory, wherein the memory is configured to store computer instructions that, when executed by the processor, cause the health data management device to perform the method according to claim 7.

24. A non-transitory computer-readable storage medium, wherein the computer-readable storage medium has stored thereon computer program instructions that, when run on a processor, cause the processor to perform the health data management method according to claim 7.

25. A non-transitory computer-readable storage medium, wherein the computer-readable storage medium has stored thereon computer program instructions that, when run on a processor, cause the processor to perform the health data management method according to claim 10.
